(19) United States
(12) Patent Application Publication
(10) Pub. No.: US 2024/0099985 A1
(43) Pub. Date: Mar. 28, 2024

(54) LIQUID CRYSTAL-INFUSED POROUS SURFACES AND METHODS OF MAKING AND USE THEREOF

(71) Applicant: Ohio State Innovation Foundation, Columbus, OH (US)

(72) Inventors: Xiaoguang WANG, Powell, OH (US); Yang XU, Columbus, OH (US); Adil RATHER, Columbus, OH (US)

(21) Appl. No.: 18/274,353

(22) PCT Filed: Jan. 27, 2022
(86) PCT No.: PCT/US2022/014006

§ 371 Date: Jul. 26, 2023

Related U.S. Application Data
(60) Provisional application No. 63/143,501, filed on Jan. 29, 2021.

(51) Int. Cl. A61K 9/70; B01L 3/00; B01L 7/00; C02F 1/32; C02F 1/72

(52) U.S. Cl. CPC A61K 9/7007; B01L 3/502715; B01L 7/52; C02F 1/32; C02F 1/725; B08B 17/02

(57) ABSTRACT

Disclosed herein are liquid crystal-infused porous surfaces and methods of making and use thereof.

### CROSS-REFERENCE TO RELATED APPLICATIONS

This application claims the benefit of priority to U.S. Provisional Application No. 63/143,501, filed Jan. 29, 2021, which is hereby incorporated herein by reference in its entirety.

### BACKGROUND

Stimuli-responsive functional surfaces have shown great promise for a wide range of applications. Nature-inspired micro/nanostructured hydrophobic (e.g., superhydrophobic) surfaces achieve excellent water-repellency. When used in actual processes, however, superhydrophobic surfaces are easily fouled by oily contaminants. This limitation can be remedied by lubricating the micro/nanostructured surface with a chemically matched lubrication film to obtain so-called slippery liquid-infused porous surfaces (SLIPS). Millimeter-sized water droplets on SLIPS are extraordinarily mobile with exceedingly low sliding angles (≤2°). Although SLIPS have shown anti-biofouling and anti-icing properties, current SLIPS rely exclusively on isotropic lubricants such as silicone oils and fluorinated oils, which inherently lack both long-range positional and orientational order. The effect of molecular order in complex and structured fluids on the property of SLIPS, however, remains unknown.

Thermotropic liquid crystals adopt a rich palette of mesophases with intrinsic positional and orientational order of constituent molecules, which enable a broad range of functional and responsive systems based on water-liquid crystal (or water-liquid crystal polymer) interfaces. Liquid crystals are a particularly promising class of anisotropic structured fluids that can offer unprecedent complexities and functionalities to SLIPS. However, past studies have reported water droplet-induced dewetting of liquid crystal films coated on conventional flat hydrophobic surfaces. Dewetting of liquid crystal films by water has been reported at silane-functionalized surfaces, and water droplets became pinned on liquid crystal-coated azlactone-functionalized surfaces (e.g., ˜10° sliding angle for a 10 μL water droplet). This has precluded the exploration of the role of hierarchical assembly of mesogens in liquid crystal mesophases on the slipperiness of liquid crystal surfaces towards water droplets.

The compositions and methods discussed herein address these and other needs.

### SUMMARY

In accordance with the purposes of the disclosed compositions and methods as embodied and broadly described herein, the disclosed subject matter relates to liquid crystal-infused porous surfaces and methods of making and use thereof.

Additional advantages of the disclosed compositions, methods, and devices will be set forth in part in the description which follows, and in part will be obvious from the description. The advantages of the disclosed compositions, methods, and devices will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1 is a schematic illustration (left) and corresponding polarized light micrograph (right) of a 1 μL water droplet on a nematic 8CB-infused porous polyRM257 surface (top view). The white double-headed arrow indicates the orientations of crossed polarizers. Scale bar, 200 μm. Inset is a conoscopic image confirming homeotropic alignment of 8CB at air-8CB surface.

FIG. 2 is a color-interferometry photograph of a water droplet on a nematic 8CB surface. Dashed red region indicates the surface of the water droplet exposed to diffuse white light. The droplet volume is 3 μL. Scale bar, 500 μm.

FIG. 3 shows the liquid crystal mesophase-dependent slipperiness of liquid crystal-infused porous surfaces. Specifically, FIG. 3 shows the sliding angle (left axis) and apparent advancing contact angle (θ_{adv}, right axis) of a 3 μL water droplet on the surface of 8CB in different mesophases. Error bars represent standard deviations and n=3 for each data point.

FIG. 4 is a representative force diagram (F_{d} versus time) of a 3 μL water droplet moving at smectic A and nematic 8CB surfaces. Inset in FIG. 4 shows data in the range of 0.4-0.8 μN.

FIG. 5 is a plot showing F_{dynamic} acting on 3 μL water droplets moving at speeds of 0.1-1 mm/s at nematic 8CB surface. Error bars represent standard deviations and n=3 for each data point.

FIG. 6 shows that F_{dynamic} follows LLD theory predicted by equation (3). The line fits the data with η of ˜20 cP. Error bars represent standard deviations and n=3 for each data point.

FIG. 7 shows the smectic A-nematic transition temperature of azobenzene-doped 8CB with and without 365 nm-wavelength UV exposure. The horizontal dashed line indicates the substrate temperature set in other experiments in FIG. 8-FIG. 10. 8CB is doped with azobenzene at 1 wt % based on 8CB. Error bars represent standard deviations and n=3 for each data point.

FIG. 8 shows the optical manipulation of droplet movement at liquid crystal surfaces. Specifically, FIG. 8 is a plot and sequential photographs showing displacement of 3 μL water droplets at an inclined 8CB surface upon periodic UV radiation. Inset shows UV-dependent droplet sliding velocity (U). Blue and orange markers indicate the data with and without UV radiation, respectively. The tilting angle of the 8CB surface is 3°. Scale bars are 2 mm.

FIG. 9 is a series of photographs showing manipulation of 3 μL water droplets' positions at an inclined 8CB surface through patterned UV exposure. The water droplets were stained with dyes for better imaging. The tilting angle of the 8CB surface is 30°. Scale bars are 5 mm.

FIG. 10 is a series of photographs showing reversible erasing and writing of UV radiation patterns to program the path of 3 μL water droplet sliding down an inclined 8CB surface. White dashed arrows indicate the trajectory of water droplets at the inclined 8CB surface. The tilting angle of the 8CB surface is 30°. Scale bars are 5 mm.

FIG. 11 shows the release profile of automatic release of nano-sized ethyl orange aggregates at nematic 8CB surfaces.

FIG. 12 shows the UV-visible spectra of automatic release of nano-sized ethyl orange aggregates at nematic 8CB surfaces corresponding to FIG. 11.

FIG. 13 is a series of sequential photographs showing the thermally triggered release of encapsulated aqueous ethyl orange microdroplets to a 10 μL water droplet at an 8CB surface. Black arrows indicate concentration of cargo microdroplets in the wetting ridge of the water droplet. Scale bars are 5 mm.

FIG. 14 shows the activated release of cargos to water droplets on liquid crystal surfaces. Specifically, FIG. 14 is a plot showing release of ethyl orange microdroplets triggered thermally (crosses), by the presence of 10 mM Ca²⁺ (circles), and by exposure to UV (triangles). In the UV-triggered release experiment, UV radiation caused a nematic-isotropic transition of 1 wt % azobenzene-doped 8CB at 38° C. 1 cm 2 of the 8CB surface contains ˜2.6 mg of loaded ethyl orange aqueous microdroplets.

FIG. 15 is a plot quantifying release of ethyl orange microdroplets triggered by different phase transitions of 8CB. Each temperature was equilibrated for 10 min. Error bars represent standard deviations and n=3 for each data point

FIG. 16 shows the calculated net force (Fₙₑₜ) acting on a 10 μm-in-diameter cargo microdroplet encapsulated in an liquid crystal surface as a function of surface-to-surface distance between the cargo microdroplet and the millimeter-sized water droplet.

FIG. 17 is a schematic illustration of principal radii of curvature at liquid crystal surface. R₁ and R₂ are the two principal radii of curvature.

FIG. 18 is a plot of calculated forces acting on cargo microdroplets as a function of the surface-to-surface distance (x) of the cargo microdroplet and a water droplet on the surface, where the cargo is encapsulated in nematic surface. The cargo microdroplet contains 5 mM SDS, and the radii of cargo microdroplets and water droplets at the liquid crystal surface are 5 μm and 1.75 mm, respectively.

FIG. 19 is a plot of the calculated forces acting on cargo microdroplets as a function of the surface-to-surface distance (x) of the cargo microdroplet and a water droplet on the surface, where the cargo is encapsulated in isotropic surface. The cargo microdroplet contains 5 mM SDS, and the radii of cargo microdroplets and water droplets at the liquid crystal surface are 5 μm and 1.75 mm, respectively.

FIG. 20 shows the calculated forces acting on cargo microdroplets as a function of the surface-to-surface distance (x) of the cargo microdroplet and a water droplet on the surface, where the cargo is encapsulated in nematic surface. The millimeter-sized droplet contains 10 mM Ca²⁺.

FIG. 21 shows the calculated forces acting on cargo microdroplets encapsulated in flat isotropic liquid crystal surface under bulk pure water as a function of their distance to the bulk water-liquid crystal surface. The cargo microdroplet contains 5 mM SDS, and the radii of cargo microdroplets and water droplets at the liquid crystal surface are 5 μm and 1.75 mm, respectively.

FIG. 22 is a series of sequential photographs showing activated loading and release of Rhodamine B-doped glycerol at 8CB surface. The volume of Rhodamine B-doped glycerol droplet and aqueous droplets are 30 and 10 μL, respectively. Scale bars are 5 mm. Insets show the fluorescence micrographs of nematic-isotropic transition-induced phase separation of glycerol and 8CB. Scale bars of insets are 20 μm.

FIG. 23 shows the release profile showing automatic release of Rhodamine B-doped glycerol to water droplets at isotropic 8CB surfaces. Glycerol is doped with 0.05 wt % Rhodamine B (RhB).

FIG. 24 shows the UV-visible spectra corresponding to FIG. 23 showing automatic release of Rhodamine B-doped glycerol to water droplets at isotropic 8CB surfaces. Glycerol is doped with 0.05 wt % Rhodamine B (RhB).

FIG. 25 is a plot showing the mass of Rhodamine B remaining in the nematic 8CB surface during the cargo loading (circles) and release process (crosses).

FIG. 26 is a plot showing the reversibility of loading and release of Rhodamine B-doped glycerol by 8CB surfaces. Error bars represent standard deviations and n=3 for each data point.

FIG. 27 illustrates the degradation and detoxification of organics in water by liquid crystal surfaces. Specifically, FIG. 27 is a series of sequential photographs showing thermally triggered release of TiO₂ nanoparticles and subsequent TiO₂-mediated photocatalytic degradation of Rhodamine B (RhB) in 10 μL water droplets by 365 nm-wavelength UV exposure. 1 wt % of TiO₂ in 0.8 mM non-ionic surfactant Brij 97 aqueous solution was dispersed in 8CB and released by a thermally triggered nematic-isotropic transition. Scale bars are 5 mm.

FIG. 28 shows the UV-visible absorbance of TiO₂-mediated photocatalytic degradation of Rhodamine B at the 8CB surface from FIG. 27. Insets show the sequential photographs of TiO₂-mediated photocatalytic degradation of Rhodamine B in a 10 μL water droplet.

FIG. 29 shows the reaction kinetics of TiO₂-mediated photocatalytic degradation of Rhodamine B at the 8CB surface from FIG. 27. Error bars represent standard deviations and n=3 for each data point.

FIG. 30 shows the photocatalytic degradation of various organic by TiO₂-loaded liquid crystal surfaces. Sequential photographs showing TiO₂-mediated photocatalytic degradation of water-soluble dye aqueous droplets. 1 wt % of TiO₂ in water was dispersed in 8CB and released by nematic-isotropic transition. The concentration of each dye was 0.6 mM. Scale bars, 5 mm.

FIG. 31 is a plot showing the reusability of TiO₂-loaded 8CB surfaces for Rhodamine B degradation. Error bars represent standard deviations and n=3 for each data point.

FIG. 32 is a plot showing F_{static} and F_{dynamic} acting on 3 μL water droplets of different concentrations of Pb 2+. Error bars represent standard deviations and n=3 for each data point.

FIG. 33 shows that F_{dynamic} follows LLD theory predicted by equation (3) for Pb²⁺ aqueous droplets. The line fits the data with η of ˜20 cP.

FIG. 34 is a photograph of 3 μL water and Pb²⁺ droplets sliding at inclined Na₂S-loaded nematic 8CB surfaces. The surface tilting angle is 10°, measured from the horizontal.

FIG. 35 is a plot of U for the 3 μL water and Pb²⁺ droplets sliding at inclined Na₂S-loaded nematic 8CB surfaces shown in FIG. 34. The surface tilting angle is 10°, measured from the horizontal.

FIG. 36 shows the sensing of heavy metal ions in water by liquid crystal surfaces. Specifically, FIG. 36 shows F_{static} of 3 μL aqueous droplets containing a variety of 50 mM heavy metal ions at Na₂S-loaded nematic 8CB surfaces. Inset is the corresponding photograph of heavy metal ion aqueous droplets at the Na₂S-loaded 8CB surface. Scale bar is 10 mm. Error bars represent standard deviations and n=3 for each data point.

FIG. 37 shows the removal of heavy metal ions in water by liquid crystal surfaces. Specifically, FIG. 37 shows the concentration of leftover heavy metal ions in water droplets after removal by Na₂S-loaded 8CB surfaces. Inset shows the corresponding removal efficiency of heavy metal ions. The initial heavy metal ion concentration in water droplets is 10 mM. Error bars represent standard deviations and n=3 for each data point.

FIG. 38 is a series of representative photographs of a capillary tube at equilibrium (0 μL) and when deflected due to the weight of the droplet (2 and 3 μL water droplets) from equilibrium position.

FIG. 39 shows the calibration of capillary tube used in force measurement. Specifically, FIG. 39 shows the droplet weight F_{g} as a function of deflection from equilibrium position Δx for the system shown in FIG. 38. The calibration curve (force measurement) is used to determine the force constant of the capillary tube using Hooke's law. The slope of the line is 17.3 mN/m, which corresponds to the spring constant k of the cantilever.

FIG. 40 shows a dispersion of nano-sized ethyl orange aggregates in nematic 8CB. Crossed double-headed arrows indicate the orientations of crossed polarizers. Single double-headed arrow indicates the orientation of rubbing direction of polyimide-coated glass substrates. Scale bar is 10 μm.

FIG. 41 is a polarized light micrograph of ethyl orange aqueous microdroplets in nematic 8CB. Crossed double-headed arrows indicate the orientations of crossed polarizers. Single double-headed arrow indicates the orientation of rubbing direction of polyimide-coated glass substrates. Scale bar is 10 μm.

FIG. 42 is a schematic illustration of ethyl orange aqueous microdroplets in nematic 8CB.

FIG. 43 is a series of micrographs of apparent Oath, and measured contact angle hysteresis of water droplets on 8CB surfaces captured at 15° C., 25° C., 35° C. and 45° C., corresponding to crystal (upper left), smectic A (upper right), nematic (lower left), and isotropic (lower right) phases, respectively. The volume of each droplet was 3 μL. Scale bars are 500 μm.

FIG. 44 shows the measured contact angle hysteresis of water droplets on 8CB surfaces for different phases of 8CB. These were calculated using equation (S3) from measurements, where data was obtained from experiments performed at 15° C., 25° C., 35° C. and 45° C., corresponding to crystal, smectic A, nematic, and isotropic phases, respectively. The volume of each water droplet used in this experiment was 3 μL. Error bars represent standard deviations and n=3 for each data point.

FIG. 45 shows the release of encapsulated ethyl orange cargos at nematic 8CB surfaces. More specifically, FIG. 45 shows the kinetics of release of ethyl orange aqueous microdroplets activated thermally, by Ca²⁺, and by UV. In thermal-activated release, the temperature was increased from 35 to 45° C. In charge-induced release, the concentration of Ca²⁺ was 10 mM. In UV-triggered release, 8CB was doped with azobenzene at 1 wt % based on 8CB.

FIG. 46 shows the UV-visible spectra of release of ethyl orange aqueous microdroplets activated thermally corresponding to FIG. 45. In thermal-activated release, the temperature was increased from 35 to 45° C.

FIG. 47 shows the UV-visible spectra of release of ethyl orange aqueous microdroplets activated by Ca²⁺ corresponding to FIG. 45. In charge-induced release, the concentration of Ca²⁺ was 10 mM.

FIG. 48 shows the UV-visible spectra of release of ethyl orange aqueous microdroplets activated by UV corresponding to FIG. 45. In UV-triggered release, 8CB was doped with azobenzene at 1 wt % based on 8CB.

FIG. 49 is a fluorescence micrograph of the condensed glycerol droplets at a nematic 8CB surface. Scale bar is 20 μm. Glycerol is doped with 0.05 wt % Rhodamine B (RhB).

FIG. 50 is the size distribution corresponding to FIG. 49 of the condensed glycerol droplets at a nematic 8CB surface. Percentages were obtained by analyzing 202 droplets in 3 independent experiments. Glycerol is doped with 0.05 wt % Rhodamine B (RhB).

FIG. 51 shows the release profile showing SDS-activated release of Rhodamine B-doped glycerol to water droplets at a nematic 8CB surface. Glycerol is doped with 0.05 wt % Rhodamine B (RhB).

FIG. 52 shows the UV-visible spectra corresponding FIG. 51 showing SDS-activated release of Rhodamine B-doped glycerol to water droplets at a nematic 8CB surface. Glycerol is doped with 0.05 wt % Rhodamine B (RhB).

FIG. 53 is a series of photographs showing in-situ loading and release of Rhodamine B-doped glycerol at nematic 8CB surface. Scale bars are 5 mm. Glycerol is doped with 0.05 wt % Rhodamine B (RhB).

FIG. 54 shows the apparent Oath, and contact angle hysteresis of water droplets with different concentrations of Pb²⁺ at 8CB surface. Plot illustrating the apparent θ_{adv} and contact angle hysteresis of water droplets with different concentrations of Pb²⁺. Error bars represent standard deviations for n=3 measurements. The volume of each droplet was 3 μL.

### DETAILED DESCRIPTION

The compositions, methods, and devices described herein may be understood more readily by reference to the following detailed description of specific aspects of the disclosed subject matter and the Examples included therein.

Before the present compositions, methods, and devices are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings.

Throughout the description and claims of this specification, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Disclosed herein are liquid crystal-infused porous surfaces comprising a porous polymer layer having a surface, wherein the porous polymer layer comprises a continuous phase permeated by a plurality of pores, and wherein the continuous phase comprises a liquid crystal polymer; and an anisotropic lubricant infused within and over the porous polymer layer, such that that the anisotropic lubricant at least partially fills the plurality of pores and forms a film on the surface of the porous polymer layer, wherein the anisotropic lubricant comprises thermotropic liquid crystal mesogen; wherein the porous polymer layer without the anisotropic lubricant has a first total interfacial energy when wetted with water; wherein: when an aqueous droplet (e.g., comprising water and optionally other species) is disposed on the film of the anisotropic lubricant, then the liquid crystal-infused porous surface has a second total interfacial energy, and in the absence of the aqueous droplet, then the liquid crystal-infused porous surface has a third total interfacial energy; wherein the first total interfacial energy is greater than the second total interfacial energy; and wherein the first total interfacial energy is greater than the third total interfacial energy.

In some examples, an aqueous droplet placed disposed on the film of the anisotropic lubricant only contacts the anisotropic lubricant (e.g., the aqueous droplet does not contact the porous polymer layer). In some examples, the liquid crystal-infused porous surfaces are stable against dewetting by an aqueous droplet placed disposed on the film of the anisotropic lubricant. In some examples, the anisotropic lubricant can at least partially wrap around (e.g., at least partially encapsulate) an aqueous droplet placed disposed on the film of the anisotropic lubricant.

In some examples, the liquid crystal-infused porous surface is disposed on a substrate. Examples of suitable substrates include, but are not limited to, polymers (e.g., porous polymers), glass fibers, glass, quartz, silicon, nitrides (e.g., silicon nitride), a ceramic, a fabric (e.g., cotton), a rubber, a metal (e.g., aluminum foil, steel, tin), a cellulosic substrate (e.g., wood), and combinations thereof. In some examples, the substrate comprises glass.

The liquid crystal polymer can comprise any suitable liquid crystal polymer. For examples, the liquid crystal polymer can be derived from 1,4-Bis-[4-(3-acryloyloxypropyloxy)benzoyloxy]-2-methylbenzene (RM257), 4-(6-acryloxy-hex-1-yl-oxy) phenyl 4-(hexyloxy) benzoate, 4-methoxybenzoic acid 4-(6-acryloyloxyhexyloxy) phenyl ester 4″-acryloyloxybutyl 2,5-di(4′-butyloxybenzoyloxy) benzoate, or combinations thereof. In some examples, the liquid crystal polymer can be derived from 1,4-Bis-[4-(3-acryloyloxypropyloxy)benzoyloxy]-2-methylbenzene (RM257).

In some examples, the porous polymer layer infused with the anisotropic lubricant has an average thickness of 100 nanometers (nm) or more (e.g., 110 nm or more, 120 nm or more, 130 nm or more, 140 nm or more, 150 nm or more, 175 nm or more, 200 nm or more, 225 nm or more, 250 nm or more, 300 nm or more, 350 nm or more, 400 nm or more, 450 nm or more, 500 nm or more, 600 nm or more, 700 nm or more, 800 nm or more, 900 nm or more, 1 micrometer (micron, μm) or more, 2 μm or more, 3 μm or more, 4 μm or more, 5 μm or more, 10 μm or more, 15 μm or more, 20 μm or more, 25 μm or more, 30 μm or more, 40 μm or more, 50 μm or more, 75 μm or more, 100 μm or more, 125 μm or more, 150 μm or more, 175 μm or more, 200 μm or more, 250 μm or more, 300 μm or more, 350 μm or more, 400 μm or more, 450 μm or more, 500 μm or more, 600 μm or more, 700 μm or more, 800 μm or more, or 900 μm or more). In some examples, the porous polymer layer infused with the anisotropic lubricant has an average thickness of 1 millimeter (mm) or less (e.g., 900 μm or less, 800 μm or less, 700 μm or less, 600 μm or less, 500 μm or less, 450 μm or less, 400 μm or less, 350 μm or less, 300 μm or less, 250 μm or less, 200 μm or less, 150 μm or less, 125 μm or less, 100 μm or less, 75 μm or less, 50 μm or less, 40 μm or less, 30 μm or less, 25 μm or less, 20 μm or less, 15 μm or less, 10 μm or less, 5 μm or less, 4 μm or less, 3 μm or less, 2 μm or less, 1 μm or less, 900 nm or less, 800 nm or less, 700 nm or less, 600 nm or less, 500 nm or less, 450 nm or less, 400 nm or less, 350 nm or less, 300 nm or less, 275 nm or less, 250 nm or less, 225 nm or less, 200 nm or less, 190 nm or less, 180 nm or less, 170 nm or less, 160 nm or less, 150 nm or less, 140 nm or less, 130 nm or less, 120 nm or less, or 110 nm or less). The average thickness of the porous polymer layer infused with the anisotropic lubricant can range from any of the minimum values described above to any of the maximum values described above. For example, the porous polymer layer infused with the anisotropic lubricant can have an average thickness of from 100 nm to 1 mm (e.g., from 100 nm to 10 μm, from 10 μm to 1 mm, from 100 nm to 1 μm, from 1 μm to 10 μm, from 10 μm to 100 μm, from 100 μm to 1 mm, from 100 nm to 900 μm, from 110 nm to 1 mm, from 110 nm to 900 μm, from 500 nm to 500 μm, from 750 nm to 500 μm, from 1 μm to 500 μm, from 1 μm to 250 μm, or from 150 μm to 175 μm). In some examples, the porous polymer layer infused with the anisotropic lubricant has an average thickness of 160 μm. The average thickness of the porous polymer layer infused with the anisotropic lubricant can be measured using methods known in the art, such as microscopy (e.g., optical microscopy, electron microscopy, etc.).

The porous polymer layer can, for example, have a porosity (e.g., pore volume percentage) of greater than 0% (e.g., 1% or more, 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, or 90% or more). In some examples, the porous polymer layer can have a porosity of 95% or less (e.g., 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less). The porosity of the porous polymer layer can range from any of the minimum values described above to any of the maximum values described above. For example, the porous polymer layer can have a porosity of from greater than 0% to 95% (e.g., from greater than 0% to 50%, from 50% to 95%, from greater than 0% to 30%, from 30% to 60%, from 60% to 95%, from 5% to 95%, from greater than 0% to 90%, or from 5% to 90%).

The film of the anisotropic lubricant can, for example, have an average thickness of 500 nm or more (e.g., 600 nm or more, 700 nm or more, 800 nm or more, 900 nm or more, 1 micrometer (micron, μm) or more, 2 μm or more, 3 μm or more, 4 μm or more, 5 μm or more, 10 μm or more, 15 μm or more, 20 μm or more, 25 μm or more, 30 μm or more, 40 μm or more, 50 μm or more, 75 μm or more, 100 μm or more, 125 μm or more, 150 μm or more, 175 μm or more, 200 μm or more, 250 μm or more, 300 μm or more, 350 μm or more, 400 μm or more, 450 μm or more, 500 μm or more, 600 μm or more, 700 μm or more, 800 μm or more, 900 μm or more, 1 millimeter (mm) or more, 2 mm or more, 3 mm or more, 4 mm or more, 5 mm or more, 10 mm or more, 15 mm or more, 20 mm or more, 25 mm or more, 30 mm or more, 40 mm or more, 50 mm or more, 75 mm or more, 100 mm or more, 125 mm or more, 150 mm or more, 200 mm or more, 250 mm or more, 300 mm or more, 350 mm or more, 400 mm or more, or 450 mm or more). In some examples, the film of the anisotropic lubricant can have an average thickness of 500 millimeters (mm) or less (e.g., 450 mm or less, 400 mm or less, 350 mm or less, 300 mm or less, 250 mm or less, 200 mm or less, 150 mm or less, 125 mm or less, 100 mm or less, 75 mm or less, 50 mm or less, 40 mm or less, 30 mm or less, 25 mm or less, 20 mm or less, 15 mm or less, 10 mm or less, 5 mm or less, 4 mm or less, 3 mm or less, 2 mm or less, 1 mm or less, 900 μm or less, 800 μm or less, 700 μm or less, 600 μm or less, 500 μm or less, 450 μm or less, 400 μm or less, 350 μm or less, 300 μm or less, 250 μm or less, 200 μm or less, 150 μm or less, 125 μm or less, 100 μm or less, 75 μm or less, 50 μm or less, 40 μm or less, 30 μm or less, 25 μm or less, 20 μm or less, 15 μm or less, 10 μm or less, 5 μm or less, 4 μm or less, 3 μm or less, 2 μm or less, 1 μm or less, 900 nm or less, 800 nm or less, 700 nm or less, or 600 nm or less). The average thickness of the film of anisotropic lubricant can range from any of the minimum values described above to any of the maximum values described above. For example, the film of the anisotropic lubricant can have an average thickness of from 500 nm to 500 mm (e.g., from 500 nm to 1 μm, from 1 μm to 500 μm, from 500 μm to 1 mm, from 1 mm to 500 mm, from 500 nm to 400 mm, from 600 nm to 500 mm, from 600 nm to 500 mm, from 500 nm to 250 mm, from 500 nm to 1 mm, from 500 nm to 750 μm, from 500 nm to 500 μm, from 750 nm to 500 μm, from 1 μm to 500 μm, from 1 μm to 250 μm, or from 100 μm to 150 μm). In some examples, the film of the anisotropic lubricant can have an average thickness of 130 μm. The average thickness of the film of the anisotropic lubricant can be measured using methods known in the art, such as microscopy (e.g., optical microscopy, fluorescence microscopy, etc.) or by measuring weight of the anisotropic lubricant and then dividing by the mass density of the anisotropic lubricant and the total surface area.

The thermotropic liquid crystal mesogen can comprise any suitable thermotropic liquid crystal mesogen. For example, the thermotropic liquid crystal mesogen comprises 4-cyano-4′-n-pentyl-biphenyl (5CB), 4-cyano-4′-n-heptyl-biphenyl (7CB), 4′-octyl-4-biphenylcarbonitrile (8CB), 4-cyano-4′-oxyoctyl-biphenyl (8OCB), 4-cyano-4′-n-pentyl-terphenyl (5CT), E7 (a nematic liquid crystal mixture containing cyanobiphenyl and cyanoterphenol components commercially available from Merck), (S)-4-Cyano-4′-(2-methylbutyl)biphenyl (CB15), or a combination thereof. In some examples, the thermotropic liquid crystal mesogen comprises 8CB.

In some examples, the anisotropic lubricant cam comprise the thermotropic liquid crystal mesogen and can further comprise a dopant. For example, the anisotropic lubricant cam comprise the thermotropic liquid crystal mesogen and can further comprise a chiral dopant, such that the liquid crystal comprises a chiral liquid crystal. Examples of chiral dopants include, but are not limited to, 4-(1-methylheptyloxycarbonyl)phenyl-4-hexyloxybenzoate (S-811/R-811).

The thermotropic liquid crystal mesogen, in some examples, has: a crystal mesophase when the thermotropic liquid crystal mesogen is at a temperature that is less than a first transition temperature, wherein when the thermotropic liquid crystal mesogen is in the crystal mesophase the thermotropic liquid crystal mesogen has long range orientational order and three dimensional positional order; a smectic mesophase when the thermotropic liquid crystal mesogen is at a temperature greater than the first transition temperature and less than a second transition temperature, wherein the second transition temperature is greater than the first transition temperature, and wherein the smectic mesophase has long range orientational order and at least unidirectional positional order; a nematic mesophase when the thermotropic liquid crystal mesogen is at a temperature greater than the second transition temperature and less than a third transition temperature, wherein the third transition temperature is greater than the second transition temperature, and wherein the nematic mesophase has long range orientational order and no positional order; and an isotropic mesophase when the thermotropic liquid crystal mesogen is at a temperature above a second transition temperature, and wherein the isotropic mesophase has no orientational order and no positional order.

In some examples, an aqueous droplet having a volume placed on the film of the anisotropic lubricant is: pinned when the thermotropic liquid crystal mesogen is in the crystal mesophase or the smectic mesophase; and mobile when the thermotropic liquid crystal mesogen is in the nematic mesophase or the isotropic mesophase; such that: when the thermotropic liquid crystal mesogen is in the crystal mesophase or the smectic mesophase, then liquid crystal-infused porous surface is in a stick-slip mode; and when the thermotropic liquid crystal mesogen is in the nematic mesophase or the isotropic mesophase, the liquid crystal-infused porous surface is in a slippery mode.

As used herein, an aqueous droplet placed on the film of the anisotropic lubricant is “pinned” when the static friction force is greater than the portion of the gravitational force of the aqueous droplet along the liquid crystal-infused porous surface when the liquid crystal-infused porous surface is titled at an angle below the sliding angle. As used herein, an aqueous droplet placed on the film of the anisotropic lubricant is “mobile” when the static friction force is less than or equal to the gravitational force when the liquid crystal-infused porous surface is titled at an angle that is greater than or equal to the sliding angle. The angle at which the liquid crystal-infused porous surface must be titled in order to cause an aqueous droplet placed on the film of the anisotropic lubricant to move is referred to herein as the “sliding angle.” An aqueous droplet placed on the film of the anisotropic lubricant with a sliding angle of 10 degrees or more (e.g., 15 degrees or more, 20 degrees or more, 25 degrees or more, 30 degrees or more, or 35 degrees or more) is considered pinned herein. An aqueous droplet placed on the film of the anisotropic lubricant with a sliding angle less than 10 degrees (e.g., 5 degrees or less, 4 degrees or less, 3 degrees or less, 2 degrees or less, or 1 degree or less) is considered mobile herein.

The volume of the aqueous droplet can affect the sliding angle. For example, aqueous droplets with larger volumes can have smaller sliding angles, and vice versa. In general, for a perfect slippery surface, the sliding angle of a 2-3 μL aqueous droplet is 2-3 degrees.

The aqueous droplet can, for example, have a volume of 0.1 microliters (4) or more (e.g., 0.2 μL or more, 0.3 μL or more, 0.4 μL or more, 0.5 μL or more, 0.75 μL or more, 1 μL or more, 1.25 μL or more, 1.5 μL or more, 1.75 μL or more, 2 μL or more, 2.5 μL or more, 3 μL or more, 3.5 μL or more, 4 μL or more, 4.5 μL or more, 5 μL or more, 6 μL or more, 7 μL or more, 8 μL or more, 9 μL or more, 10 μL or more, 11 μL or more, 12 μL or more, 13 μL or more, 14 μL or more, 15 μL or more, 20 μL or more, 25 μL or more, 30 μL or more, 35 μL or more, 40 μL or more, 45 μL or more, 50 μL or more, 60 μL or more, 70 μL or more, 80 μL or more, 90 μL or more, 100 μL or more, 125 μL or more, 150 μL or more, 175 μL or more, 200 μL or more, 250 μL or more, 300 μL or more, 350 μL or more, 400 μL or more, 450 μL or more, 500 μL or more, 600 μL or more, 700 μL or more, 800 μL or more, 900 μL or more, 1 milliliters (mL) or more, 2 mL or more, 3 mL or more, 4 mL or more, 5 mL or more, 10 mL or more, 15 mL or more, 20 mL or more, 25 mL or more, 30 mL or more, 35 mL or more, 40 mL or more, 45 mL or more, 50 mL or more, 60 mL or more, 70 mL or more, 80 mL or more, 90 mL or more, 100 mL or more, 125 mL or more, 150 mL or more, 175 mL or more, 200 mL or more, 250 mL or more, 300 mL or more, 350 mL or more, 400 mL or more, or 450 mL or more). In some examples, the aqueous droplet can have a volume of 500 milliliters (mL) or less (e.g., 450 mL or less, 400 mL or less, 350 mL or less, 300 mL or less, 250 mL or less, 200 mL or less, 175 mL or less, 150 mL or less, 125 mL or less, 100 mL or less, 90 mL or less, 80 mL or less, 70 mL or less, 60 mL or less, 50 mL or less, 45 mL or less, 40 mL or less, 35 mL or less, 30 mL or less, 25 mL or less, 20 mL or less, 15 mL or less, 10 mL or less, 5 mL or less, 4 mL or less, 3 mL or less, 2 mL or less, 1 mL or less, 900 μL or less, 800 μL or less, 700 μL or less, 600 μL or less, 500 μL or less, 450 μL or less, 400 μL or less, 350 μL or less, 300 μL or less, 250 μL or less, 200 μL or less, 175 μL or less, 150 μL or less, 125 μL or less, 100 μL or less, 90 μL or less, 80 μL or less, 70 μL or less, 60 μL or less, 50 μL or less, 45 μL or less, 40 μL or less, 35 μL or less, 30 μL or less, 25 μL or less, 20 μL or less, 15 μL or less, 14 μL or less, 13 μL or less, 12 μL or less, 11 μL or less, 10 μL or less, 9 μL or less, 8 μL or less, 7 μL or less, 6 μL or less, 5 μL or less, 4.5 μL or less, 4 μL or less, 3.5 μL or less, 3 μL or less, 2.5 μL or less, 2 μL or less, 1.75 μL or less, 1.5 μL or less, 1.25 μL or less, 1 μL or less, 0.75 μL or less, or 0.5 μL or less). The volume of the aqueous droplet can range from any of the minimum values described above to any of the maximum values described above. For example, the aqueous droplet can have a volume of from 0.1 μL to 500 mL (e.g., from 0.1 μL to 10 μL, from 10 μL to 1 mL, from 1 mL to 500 mL, from 0.1 μL to 1 μL, from 1 μL to 10 μL, from 10 μL to 100 μL, from 100 μL to 1 mL, from 1 mL to 10 mL, from 10 mL to 500 mL, from 0.5 μL to 500 mL, from 0.1 μL to 400 mL, or from 0.5 μL to 400 mL). In some examples, the aqueous droplet can have a volume of 10 microliters (4).

In some examples, the positional order of the thermotropic liquid crystal mesogen can affect the static friction of the aqueous droplet on the film of anisotropic lubricant. In some examples, external stimuli that affects the positional order of thermotropic liquid crystal can tune the mobility of aqueous droplets placed on the film of the anisotropic lubricant. In some examples, manipulation of the positional order of the thermotropic liquid crustal enables interfacial transport of liquid droplets in a programmable manner.

In some examples, the mobility of the aqueous droplet on the film of the anisotropic lubricant is temperature sensitive, as: when the temperature of the thermotropic liquid crystal mesogen is increased from a temperature below the second transition temperature to a temperature above the second transition temperature, then the liquid crystal-infused porous surface transitions from the stick-slip mode to the slippery mode, such that the aqueous droplet becomes mobile; and when the temperature of the thermotropic liquid crystal mesogen is decreased from a temperature above the second transition temperature to a temperature below the second transition temperature, then the liquid crystal-infused porous surface transitions from the slippery mode to the stick-slip mode, such that the aqueous droplet becomes pinned.

In some examples, the anisotropic lubricant further comprises a compound that can undergo photoisomerization (e.g., a photoisomerization compound), such as azobenzene (e.g., heteroaryl azobenzene), spiropyran, derivatives thereof, and combinations thereof. In some examples, the anisotropic lubricant further comprises azobenzene.

The anisotropic lubricant can, for example, comprise a photoisomerization compound in an amount of 0.1 wt % or more (e.g., 0.25 wt % or more, 0.5 wt % or more, 0.75 wt % or more, 1 wt % or more, 1.25 wt % or more, 1.5 wt % or more, 1.75 wt % or more, 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, or 9.5 wt % or more). In some examples, the anisotropic lubricant can comprise a photoisomerization compound in an amount of 10 wt % or less (e.g., 9.5 wt % or less, 9 wt % or less, 8.5 wt % or less, 8 wt % or less, 7.5 wt % or less, 7 wt % or less, 6.5 wt % or less, 6 wt % or less, 5.5 wt % or less, 5 wt % or less, 4.5 wt % or less, 4 wt % or less, 3.5 wt % or less, 3 wt % or less, 2.5 wt % or less, 2 wt % or less, 1.75 wt % or less, 1.5 wt % or less, 1.25 wt % or less, 1 wt % or less, 0.75 wt % or less, 0.5 wt % or less, or 0.25 wt % or less). The amount of the photoisomerization compound in the anisotropic lubricant can range from any of the minimum values described above to any of the maximum values described above. For example, the anisotropic lubricant can comprise the photoisomerization compounds in an amount of from 0.1 wt % to 10 wt % (e.g., from 0.1 wt % to 5 wt %, from 0.1 wt % to 10 wt %, from 0.1 wt % to 2 wt %, from 2 wt % to 4 wt %, from 4 wt % to 6 wt %, from 6 wt % to 8 wt % from 8 wt % to 10 wt %, from 0.5 wt % to 10 wt %, from 0.1 wt % to 9 wt %, or from 0.5 wt % to 9 wt %). In some examples, the anisotropic lubricant further comprises azobenzene in an amount of from 0.1 wt % to 10 wt %.

In some examples, when the photoisomerization compound is a first photoisomer, the second transition temperature has a first value; when the photoisomerization compound is a second photoisomer, the second transition temperature has a second value; the first value is greater than the second value; in the absence of a certain wavelength (e.g., a certain range of wavelengths) of light, the photoisomerization compound is the first photoisomer; and when the photoisomerization compound is exposed to the certain wavelength of light, then the photoisomerization compound isomerizes to the second photoisomer; and when the anisotropic lubricant is at a temperature above the first value and below the second value, then the mobility of the aqueous droplet on the film of the anisotropic lubricant is light sensitive as: in the absence of the certain wavelength of light, the photoisomerization compound is the first photoisomer such that the second transition temperature has the first value, the thermotropic liquid crystal mesogen is in the smectic mesophase, the liquid-crystal infused porous surface is in the stick-slip mode, and the aqueous droplet is pinned; and when the photoisomerization compound is exposed to the certain wavelength light, then the photoisomerization compound isomerizes to the second photoisomer such that the second transition temperature has the second value, the thermotropic liquid crystal mesogen is in the nematic mesophase, the liquid crystal-infused porous surface is in the slippery mode, and the aqueous droplet is mobile.

In some examples, the anisotropic lubricant further comprises azobenzene and when the azobenzene is trans-azobenzene, the second transition temperature has a first value; when the azobenzene is cis-azobenzene, the second transition temperature has a second value; the first value is greater than the second value; in the absence of UV light, the azobenzene is trans-azobenzene; and when the azobenzene is exposed to UV light, then the azobenzene isomerizes to cis-azobenzene; and when the anisotropic lubricant is at a temperature above the first value and below the second value, then the mobility of the aqueous droplet on the film of the anisotropic lubricant is UV light sensitive as: in the absence of UV light, the azobenzene is trans-azobenzene such that the second transition temperature has the first value, the thermotropic liquid crystal mesogen is in the smectic mesophase, the liquid-crystal infused porous surface is in the stick-slip mode, and the aqueous droplet is pinned; and when the azobenzene is exposed to UV light, then the azobenzene isomerizes to cis-azobenzene such that the second transition temperature has the second value, the thermotropic liquid crystal mesogen is in the nematic mesophase, the liquid crystal-infused porous surface is in the slippery mode, and the aqueous droplet is mobile.

In some examples, the liquid crystal-infused porous surface has a first portion and a second portion, wherein the first portion is selectively exposed to a certain wavelength of light and the second portion is not exposed to the certain wavelength of light, such that, when present, an aqueous droplet disposed on the first portion is mobile while an aqueous droplet disposed on the second portion is pinned. In some examples, the first portion abuts the second portion along a border, and the border defines a path for motion of an aqueous droplet, when present.

In some examples, the anisotropic lubricant further comprises a cargo. In some examples, the anisotropic lubricant further comprises a plurality of droplets comprising a cargo.

The cargo can, for example, comprise an organic species, a photocatalyst, a heavy metal ion capture species, a medicament, a drug, or a combination thereof. In some examples, the cargo comprises an organic species and the organic species comprises an organic contaminant. In some examples, the cargo comprises an organic species and organic species comprises a water soluble dye. Examples of water soluble dyes include, but are not limited to, ethyl orange, rhodamine B, methyl orange, methylene blue, and combinations thereof. In some examples, the cargo comprises a plurality of particles comprising a photocatalyst. In some examples, the photocatalyst comprises TiO₂, ZnO, CdS, WO₂, derivatives thereof, and combinations thereof. In some examples, the cargo comprises a heavy metal ion capture species comprising S²⁻.

The plurality of droplets can, for example, have an average diameter of 1 nm or more (e.g., 2 nm or more, 3 nm or more, 4 nm or more, 5 nm or more, 10 nm or more, 15 nm or more, 20 nm or more, 25 nm or more, 30 nm or more, 35 nm or more, 40 nm or more, 45 nm or more, 50 nm or more, 60 nm or more, 70 nm or more, 80 nm or more, 90 nm or more, 100 nm or more, 125 nm or more, 150 nm or more, 175 nm or more, 200 nm or more, 225 nm or more, 250 nm or more, 300 nm or more, 350 nm or more, 400 nm or more, 450 nm or more, 500 nm or more, 600 nm or more, 700 nm or more, 800 nm or more, 900 nm or more, 1 micrometer (micron, μm) or more, 2 μm or more, 3 μm or more, 4 μm or more, 5 μm or more, 10 μm or more, 15 μm or more, 20 μm or more, 25 μm or more, 30 μm or more, 35 μm or more, 40 μm or more, 45 μm or more, 50 μm or more, 60 μm or more, 70 μm or more, 80 μm or more, or 90 μm or more). In some examples, the plurality of droplets can have an average diameter of 100 μm or less (e.g., 90 μm or less, 80 μm or less, 70 μm or less, 60 μm or less, 50 μm or less, 45 μm or less, 40 μm or less, 35 μm or less, 30 μm or less, 25 μm or less, 20 μm or less, 15 μm or less, 10 μm or less, 5 μm or less, 4 μm or less, 3 μm or less, 2 μm or less, 1 μm or less, 900 nm or less, 800 nm or less, 700 nm or less, 600 nm or less, 500 nm or less, 450 nm or less, 400 nm or less, 350 nm or less, 300 nm or less, 275 nm or less, 250 nm or less, 225 nm or less, 200 nm or less, 175 nm or less, 150 nm or less, 125 nm or less, 100 nm or less, 90 nm or less, 80 nm or less, 70 nm or less, 60 nm or less, 50 nm or less, 45 nm or less, 40 nm or less, 35 nm or less, 30 nm or less, 25 nm or less, 20 nm or less, 15 nm or less, 10 nm or less, 5 nm or less, 4 nm or less, 3 nm or less, or 2 nm or less). The average diameter of the plurality of droplets can range from any of the minimum values described above to any of the maximum values described above. For example, the plurality of droplets can have an average diameter of from 1 nm to 100 μm (e.g., from 1 nm to 100 nm, from 100 nm to 100 μm, from 1 nm to 10 nm, from 10 nm to 100 nm, from 100 nm to 1 μm, from 1 μm to 10 μm, from 10 μm to 100 μm, from 1 nm to 90 μm, from 10 nm to 100 μm, or from 10 nm to 90 μm).

In some examples, the plurality of droplets comprising the cargo have an average radius, an average elastic energy, and an average a surface anchoring energy; the elastic energy is equal to the product of the average radius of the plurality of droplets and the Frank elastic constant; the average surface anchoring energy is equal to the product of the average radius of the plurality of droplets squared and the surface anchoring strength; when the thermotropic liquid crystal mesogen is in the isotropic mesophase and an aqueous droplet is placed on the film of anisotropic lubricant, at least a portion of the plurality of droplets comprising the cargo are automatically released from the anisotropic lubricant into the aqueous droplet; when an aqueous droplet is placed on the film of anisotropic lubricant and the thermotropic liquid crystal mesogen is in the nematic mesophase, at least a portion of the plurality of droplets comprising the cargo are automatically released from the anisotropic lubricant into the aqueous droplet when the surface anchoring energy is less than or equal to the elastic energy; and when the aqueous droplet is placed on the film of anisotropic lubricant and the thermotropic liquid crystal mesogen is the in the nematic mesophase, substantially none of the plurality of droplets comprising the cargo are released from the anisotropic lubricant into the aqueous droplet when the surface anchoring energy is greater than the elastic energy.

In some examples, the surface anchoring energy is less than or equal to the elastic energy when the average radius of the plurality of droplets comprising the cargo is less than or equal to the quotient of the Frank elastic constant and the surface anchoring strength. In some examples, the surface anchoring energy is less than or equal to the elastic energy when the average radius of the plurality of droplets comprising the cargo is 1 micrometer or less (e.g., 900 nm or less, 800 nm or less, 700 nm or less, 600 nm or less, 500 nm or less, 450 nm or less, 400 nm or less, 350 nm or less, 300 nm or less, 250 nm or less, 225 nm or less, 200 nm or less, 175 nm or less, 150 nm or less, 125 nm or less, 100 nm or less, 90 nm or less, 80 nm or less, 70 nm or less, 60 nm or less, 50 nm or less, 45 nm or less, 40 nm or less, 35 nm or less, 30 nm or less, 25 nm or less, 20 nm or less, 15 nm or less, 10 nm or less, 5 nm or less, 4 nm or less, 3 nm or less, 2 nm or less, or 1 nm or less).

In some examples, the surface anchoring energy is greater than the elastic energy when the average radius of the plurality of droplets comprising the cargo is greater than the quotient of the Frank elastic constant and the surface anchoring strength. In some examples, the surface anchoring energy is greater than the elastic energy when the average radius of the plurality of droplets comprising the cargo is greater than 1 micrometer (e.g., 2 μm or more, 3 μm or more, 4 μm or more, 5 μm or more, 10 μm or more, 15 μm or more, 20 μm or more, 25 μm or more, 30 μm or more, 35 μm or more, 40 μm or more, 45 μm or more, 50 μm or more, 60 μm or more, 70 μm or more, 80 μm or more, 90 μm or more, or 100 μm or more).

In some examples, the release of at least a portion of the plurality droplets comprising the cargo is temperature sensitive as: when the temperature of the thermotropic liquid crystal mesogen is increased from a temperature below the third transition temperature to a temperature above the third transition temperature, then the thermotropic liquid crystal transitions to the isotropic mesophase, such that at least a portion of the plurality of droplets comprising the cargo are automatically released into an aqueous droplet present on the film of anisotropic lubricant.

In some examples, the anisotropic lubricant further comprises a photoisomerization compound, and when the photoisomerization compound is a first photoisomer, the second transition temperature has a first value; when the photoisomerization compound is a second photoisomer, the second transition temperature has a second value; the first value is greater than the second value; in the absence of a certain wavelength of light, the photoisomerization compound is the first photoisomer; and when the photoisomerization compound is exposed to the certain wavelength of light, then the photoisomerization compound isomerizes to the second photoisomer; and when the anisotropic lubricant is at a temperature above the first value and below the second value, then the mobility of the aqueous droplet on the film of the anisotropic lubricant is light sensitive as: in the absence of the certain wavelength of light, the photoisomerization compound is the first photoisomer such that the second transition temperature has the first value, the thermotropic liquid crystal mesogen is in the smectic mesophase, the liquid-crystal infused porous surface is in the stick-slip mode, and the aqueous droplet is pinned; and when the photoisomerization compound is exposed to the certain wavelength of light, then the photoisomerization compound isomerizes to the second photoisomer such that the second transition temperature has the second value, the thermotropic liquid crystal mesogen is in the nematic mesophase, the liquid crystal-infused porous surface is in the slippery mode, and the aqueous droplet is mobile.

In some examples, the anisotropic lubricant further comprises a photoisomerization compound, and when the photoisomerization compound is a first photoisomer, the third transition temperature has a first value; when the photoisomerization compound is a second photoisomer, the third transition temperature has a second value; the first value is greater than the second value; in the absence of a certain wavelength of light, the photoisomerization compound is the first photoisomer; and when the photoisomerization compound is exposed to the certain wavelength of light, then the photoisomerization compound isomerizes to the second photoisomer; and when the anisotropic lubricant is at a temperature above the first value and below the second value, then release of at least a portion of the plurality droplets comprising the cargo is light sensitive as: in the absence of the certain wavelength of light, the photoisomerization compound is the first photoisomer such that the third transition temperature has the first value, the thermotropic liquid crystal mesogen is in the nematic mesophase, and substantially none of the plurality of droplets comprising the cargo are released from the anisotropic lubricant into an aqueous droplet present on the film of anisotropic lubricant; and when the photoisomerization compound is exposed to the certain wavelength of light, then the photoisomerization compound isomerizes to the second photoisomer such that the third transition temperature has the second value, the thermotropic liquid crystal mesogen is in the isotropic mesophase, and at least a portion of the plurality of droplets comprising the cargo are released from the anisotropic lubricant into an aqueous droplet present on the film of anisotropic lubricant.

In some examples, the anisotropic lubricant further comprises azobenzene, and when the azobenzene is trans-azobenzene, the third transition temperature has a first value; when the azobenzene is cis-azobenzene, the third transition temperature has a second value; the first value is greater than the second value; in the absence of UV light, the azobenzene is trans-azobenzene; and when the azobenzene is exposed to UV light, then the azobenzene isomerizes to cis-azobenzene; and when the anisotropic lubricant is at a temperature above the first value and below the second value, then release of at least a portion of the plurality droplets comprising the cargo is UV sensitive as: in the absence of UV light, the azobenzene is trans-azobenzene such that the third transition temperature has the first value, the thermotropic liquid crystal mesogen is in the nematic mesophase, and substantially none of the plurality of droplets comprising the cargo are released from the anisotropic lubricant into an aqueous droplet present on the film of anisotropic lubricant; and when the azobenzene is exposed to UV light, then the azobenzene isomerizes to cis-azobenzene such that the third transition temperature has the second value, the thermotropic liquid crystal mesogen is in the isotropic mesophase, and at least a portion of the plurality of droplets comprising the cargo are released from the anisotropic lubricant into an aqueous droplet present on the film of anisotropic lubricant.

In some examples, when the aqueous droplet has a first zeta potential and the cargo has a second zeta potential, the release of at least a portion of the plurality droplets comprising the cargo when the thermotropic liquid crystal mesogen is in the nematic mesophase is charge sensitive, as: when the first zeta potential and the second zeta potential have the same sign, then substantially none of the plurality of droplets comprising the cargo are released from the anisotropic lubricant into the aqueous droplet; and when the first zeta potential and the second zeta potential have opposite signs, then at least a portion of the plurality of droplets comprising the cargo are released from the anisotropic lubricant into the aqueous droplet.

In some examples, the liquid crystal-infused porous surface is self-sensing and self-cleaning.

Also disclosed herein are methods of making the liquid crystal-infused porous surfaces described herein. For example, the methods can comprise contacting the porous polymer layer with an anisotropic lubricant. In some examples, the methods can further comprise making the porous polymer layer.

Also disclosed herein are methods of use of the liquid crystal-infused porous surfaces described herein.

In some examples, the mobility of the aqueous droplet on the film of the anisotropic lubricant is temperature sensitive and methods can comprise controlling and/or adjusting the temperature of the thermotropic liquid crystal mesogen, thereby controlling and/or adjusting the mobility of the aqueous droplet on the film of the anisotropic lubricant.

In some examples, the anisotropic lubricant further comprises a photoisomerization compound and the mobility of the aqueous droplet on the film of the anisotropic lubricant is light sensitive, and methods can comprise patterning the liquid crystal-infused porous surface by selectively exposing the first portion of the liquid crystal-infused porous surface to a certain wavelength of light while the second portion is not exposed to the certain wavelength of light. In some examples, the methods can further comprise erasing the patterning of the liquid crystal-infused porous surface by removing the certain wavelength of light or protecting/blocking the entire liquid crystal-infused porous surface from the certain wavelength of light. In some examples, the methods comprise reversibly patterning and erasing the pattern from liquid crystal-infused porous surface by repeating these methods.

In some examples, the anisotropic lubricant further comprises azobenzene and the mobility of the aqueous droplet on the film of the anisotropic lubricant is UV light sensitive, and methods can comprise patterning the liquid crystal-infused porous surface by selectively exposing the first portion of the liquid crystal-infused porous surface to UV light while the second portion is not exposed to the UV light. In some examples, the methods can further comprise erasing the patterning of the liquid crystal-infused porous surface by removing the UV light or protecting/blocking the entire liquid crystal-infused porous surface from the UV light. In some examples, the methods comprise reversibly patterning and erasing the pattern from liquid crystal-infused porous surface by repeating these methods.

In some examples, the anisotropic lubricant further comprises a plurality of droplets comprising a cargo and the methods of use comprise placing an aqueous droplet on the film of anisotropic lubricant when the thermotropic liquid crystal mesogen is in the isotropic mesophase, thereby releasing at least a portion of the plurality of droplets comprising the cargo into the aqueous droplet.

In some examples, the anisotropic lubricant further comprises a plurality of droplets comprising a cargo and the methods of use comprise placing an aqueous droplet on the film of anisotropic lubricant when the thermotropic liquid crystal mesogen is in the nematic mesophase and the surface anchoring energy is less than or equal to the elastic energy, thereby releasing at least a portion of the plurality of droplets comprising the cargo from the anisotropic lubricant into the aqueous droplet.

In some examples, the anisotropic lubricant further comprises a plurality of droplets comprising a cargo and the release of at least a portion of the plurality droplets comprising the cargo is temperature sensitive, and the methods of use comprise controlling and/or adjusting the temperature of the thermotropic liquid crystal mesogen, thereby releasing at least a portion of the plurality of droplets comprising the cargo into an aqueous droplet present on the film of anisotropic lubricant.

In some examples, the anisotropic lubricant further comprises a plurality of droplets comprising a cargo and the release of at least a portion of the plurality droplets comprising the cargo is light sensitive, and the methods of use comprise exposing at least a portion of the liquid crystal-infused porous surface to a certain wavelength of light, thereby releasing at least a portion of the plurality of droplets comprising the cargo from the anisotropic lubricant into an aqueous droplet present on the film of anisotropic lubricant.

In some examples, the anisotropic lubricant further comprises a plurality of droplets comprising a cargo and the release of at least a portion of the plurality droplets comprising the cargo is UV sensitive, and the methods of use comprise exposing at least a portion of the liquid crystal-infused porous surface to UV light, thereby releasing at least a portion of the plurality of droplets comprising the cargo from the anisotropic lubricant into an aqueous droplet present on the film of anisotropic lubricant.

In some examples, the methods further comprise loading the film of anisotropic lubricant with the plurality of droplets comprising the cargo. In some examples, the methods comprise reversibly loading and releasing at least a portion of the plurality of droplets comprising the cargo by repeating the appropriate methods. In some examples, the methods further comprise repeating the methods to release at least a second portion of the plurality of droplets comprising the cargo.

In some examples, the cargo comprises a photocatalyst and the aqueous droplet further comprises an organic contaminant, and the methods further comprising exposing the photocatalyst to electromagnetic radiation to thereby photocatalytically degrade the organic contaminant. In some examples, the cargo comprises a photocatalyst and the aqueous droplet further comprises a water soluble dye, and the methods further comprising exposing the photocatalyst to electromagnetic radiation to thereby photocatalytically degrade the water soluble dye.

In some examples, the anisotropic lubricant further comprises a plurality of droplets comprising a cargo and the release of at least a portion of the plurality droplets comprising the cargo is charge sensitive, and the methods of use comprise disposing an aqueous droplet onto the film of anisotropic lubricant comprising a plurality of droplets comprising a cargo, wherein the aqueous droplet has a first zeta potential and the cargo has a second zeta potential, wherein the first zeta potential and the second zeta potential have opposite signs, whereby releasing at least a portion of the plurality of droplets comprising the cargo from the anisotropic lubricant into the aqueous droplet. In some examples, the methods further comprise repeating the method to release at least a second portion of the plurality of droplets comprising the cargo.

In some examples, the aqueous droplet comprises a heavy metal ion and the cargo comprises a heavy metal ion capture species. In some examples the heavy metal ion capture species comprises S²⁻. In some examples, the heavy metal ion comprises Pb, Cd, Fe, Ag, Cu, Hg, Zn, As, or a combination thereof.

In some examples, the heavy metal ion capture species and the heavy metal ion react to form a precipitate in the aqueous droplet. In some examples, formation of the precipitate induces slowing and eventual pinning of the aqueous droplet. In some examples substantially all of the heavy metal ions react to form the precipitate. In some examples, the concentration of the heavy metal ion remaining in the aqueous droplet after the formation of the precipitate is 1 ppm or less (e.g., 0.9 ppm or less, 0.8 ppm or less, 0.7 ppm or less, 0.6 ppm or less, 0.5 ppm or less, 0.4 ppm or less, 0.3 ppm or less, 0.2 ppm or less, or 0.1 ppm or less).

In some examples, the method of use of the liquid crystal-infused porous surface comprises organic contaminant removal, heavy metal ion removal, or a combination thereof.

In some examples, the cargo comprises a medicament, a drug, or a combination thereof, such that the methods of use comprise drug delivery.

Also disclosed herein are devices, systems, and articles of manufacture comprising any of the liquid crystal-infused porous surfaces described herein. For example, also described herein are drug delivery devices comprising any of the liquid crystal-infused porous surfaces described herein. Also described herein are microfluidic devices comprising any of the liquid crystal-infused porous surfaces described herein. Also disclosed herein are smart surface reactors comprising any of the liquid crystal-infused porous surfaces described herein. Also described herein are wastewater diagnosis and treatment devices comprising any of the liquid crystal-infused porous surfaces described herein. Also described herein are methods of use of any of the liquid crystal-infused porous surfaces described herein for theranostics and/or biomedical applications.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Liquid Crystal-Infused Porous Surfaces with Molecular Order-Dependent Slipperiness and Cargo Loading/Release

Abstract. The inherent molecular order of thermotropic liquid crystals has expanded their utility beyond display technologies and into the realm of functional surfaces that can sense chemical stimuli and template polymerization. However, water-induced instability of liquid crystal films has, until now, precluded the study of how molecular order affects a liquid crystal surface's slipperiness to millimeter-sized water droplets. In this work, a nature-inspired strategy is used to stabilize liquid crystal films against this dewetting, and it was found that mesogenic positional order significantly affects the mobility of water droplets by switching between stick-slip and slippery modes. Furthermore, it was demonstrated that various stimuli, including heat, light, and charge, can tune mesogenic orientational ordering to enable programmable cargo loading and release. These results reveal that the intrinsic molecular order of liquid crystals offers unique possibilities for the design of smart surfaces that can manipulate droplet transport, as well as self-sense and self-clean pollutants in water.

Introduction. Stimuli-responsive functional surfaces have shown great promise for a wide range of applications ranging from liquid transport to oil-water separation (Liu M et al. Nat. Rev. Mater. 2017, 2, 17036). Nature-inspired micro/nanostructured hydrophobic (e.g., superhydrophobic) surfaces achieve excellent water-repellency by forcing water droplets to adapt high apparent contact angles (>)150° (Tian X et al. Science 2016, 352, 142-143; Gao N et al. Nat. Phys. 2017, 14, 191-196; de Gennes P G et al. Capillarity and Wetting Phenomena (Springer, 2004)). When used in actual processes, however, superhydrophobic surfaces are easily fouled by oily contaminants. This limitation can be remedied by lubricating the micro/nanostructured surface with a chemically matched lubrication film to obtain so-called slippery liquid-infused porous surfaces (SLIPS) (Wong T S et al. Nature 2011, 477, 443-447). Millimeter-sized water droplets on SLIPS are extraordinarily mobile with exceedingly low sliding angles (≤2°) (Smith J D et al. Soft Matter 2013, 9, 1772-1780; Li J et al. Adv. Funct. Mater. 2019, 29, 1802317; Daniel D et al. Nat. Phys. 2017, 13, 1020-1025; Chen H et al. Nature 2016, 532, 85-89; Schellenberger F et al. Soft Matter 2015, 11, 7617-7626). Although SLIPS have shown anti-biofouling (Dong Z et al. Adv Mater 2018, 30, e1803890) and anti-icing properties (Kreder M J et al. Nat. Rev. Mater. 2016, 1, 15003), current SLIPS rely exclusively on isotropic lubricants such as silicone oils and fluorinated oils, which inherently lack both long-range positional and orientational order. The effect of molecular order in complex and structured fluids on the property of SLIPS, however, remains unknown.

Thermotropic liquid crystals adopt a rich palette of mesophases with intrinsic positional and orientational order of constituent molecules (Kiernan M et al. Soft Matter Physics: An Introduction (Springer, 2003)), which enable a broad range of functional and responsive systems based on water-liquid crystal (or water-liquid crystal polymer) interfaces that are capable of sensing chemicals (Woltman S J et al. Nat. Mater. 2007, 6, 929-938; Lin I H et al. Science 2011, 332, 1297-1300), liquid transport (Lv J A et al. Nature 2016, 537, 179-184; Sengupta A et al. Soft Matter 2013, 9, 7251), particle synthesis (Wei W S et al. Nature 2019, 576, 433-436; Schwartz M et al. Adv. Mater. 2018, 30, e1707382), and activated release (Zhan Y et al. Matter, 2020, 3(3), 782-793; Kim Y K et al. Nature 2018, 557, 539-544; Guo J K et al. Adv. Sci. 2019, 6, 1900785). Liquid crystals are a particularly promising class of anisotropic structured fluids that can offer unprecedent complexities and functionalities to SLIPS. However, past studies have reported water droplet-induced dewetting of liquid crystal films coated on conventional flat hydrophobic surfaces. For example, 5CB was infused into in a 20 μm-thick gold TEM grid placed on a DMOAP-functionalized glass slide, and a 1 μL droplet of pure water was deposited on top; the water droplet-induced dewetting of liquid crystal in TEM grid. Dewetting of liquid crystal films by water has been reported at silane-functionalized surfaces (Yang Z et al. Langmuir 2010, 26, 13797-13804), and water droplets became pinned on liquid crystal-coated azlactone-functionalized surfaces (e.g., −10° sliding angle for a 10 μL water droplet (Manna U et al. Adv. Mater. 2015, 27, 3007-3012)). This has precluded the exploration of the role of hierarchical assembly of mesogens in liquid crystal mesophases on the slipperiness of liquid crystal surfaces towards water droplets.

To address this long-standing challenge, herein porous liquid crystal polymeric networks are used to stabilize thermotropic liquid crystal mesogens against dewetting by water droplets, allowing the effect of mesogenic molecular order on the slipperiness of liquid crystal surfaces to be investigated. The stimuli-responsive characteristics of the liquid crystal-infused porous surfaces developed in this work are based on one central concept: any environmental cue that alters the molecular order of liquid crystals can cause the liquid crystal surface to respond. It was observed that external stimuli, including heat and light, were able to change the positional order of the mesogens, thus tuning the mobility of water droplets at the liquid crystal surface. Importantly, reversible, in-situ loading and release of cargos can be achieved via reversible switching of the orientational order of the mesogens in the liquid crystal surface. Overall, these results reveal that both positional and orientational order of anisotropic, structured fluids can be selectively manipulated to influence both droplet mobility and cargo loading/release, thus providing a new route for design of open surface microfluidics and smart surface reactors that enable self-sensing and self-cleaning of heavy metal ions in water.

Results

Design and synthesis of liquid crystal-infused porous surfaces. Based on previous studies on SLIPS, the total interfacial energy of the porous substrate wetted by water (E_{A}) needs to be higher than those of liquid crystal-infused porous surfaces with (E₁) or without (E₂) water floating on their top to form a stable liquid crystal-infused porous surface that will not be dewetted by water (Wong T S et al. Nature 2011, 477, 443-447):

ΔE₁=E_{A}−E₁=r(γ_{LC} cos θ_{LC}−γ_{w} cos θ_{w})−γ_{w-LC}>0 (1)

ΔE₂=E_{A}−E₂=r(γ_{LC} cos θ_{LC}−γ_{w} cos θ_{w})−γ_{w}−γ_{LC}>0 (1)

in which r is the roughness factor (defined as the ratio of true surface area of the porous surface to the projected surface area), γ_{w-LC} is the interfacial tension between the liquid crystal and water, and γ_{w} and γ_{LC} represent surface tensions of water and the liquid crystal, respectively. θ_{w} and θ_{LC} are the equilibrium contact angles of water and the liquid crystal at the solid surface, respectively.

To satisfy the above criteria, polymers with similar molecular structures as liquid crystals were used to form the porous substrate (e.g., chemical matching), which stabilized the liquid crystal film against dewetting by water. Based on experimental measurements, both ΔE₁ and ΔE₂>0 were calculated for a combination of the liquid crystal polymer polyRM257 and the unreactive liquid crystal 8CB (see Supplementary Materials). In the first set of experiments, a mixture of reactive RM257 (10 wt %) and 8CB (90 wt %) was photopolymerized to form 8CB-swelled polyRM257 nanoporous structures on silane-functionalized glass substrates (FIG. 1). Afterwards, a ˜130 μm-thick layer of pure 8CB was drop-casted on these structures to obtain 8CB-infused porous polyRM257 surfaces.

Liquid crystal mesophase-dependent slipperiness of liquid crystal surfaces. As temperature increases, 8CB undergoes phase transitions from crystal to smectic A to nematic, and finally to isotropic phase, which are all characterized by different types and degrees of molecular order (Kiernan M et al. Soft Matter Physics: An Introduction (Springer, 2003)). First, the behavior of water droplets deposited on a surface infused with nematic 8CB, where the constituent molecules have long-range orientational order but no positional order (e.g., they self-align with centers of mass randomly distributed), was characterized.

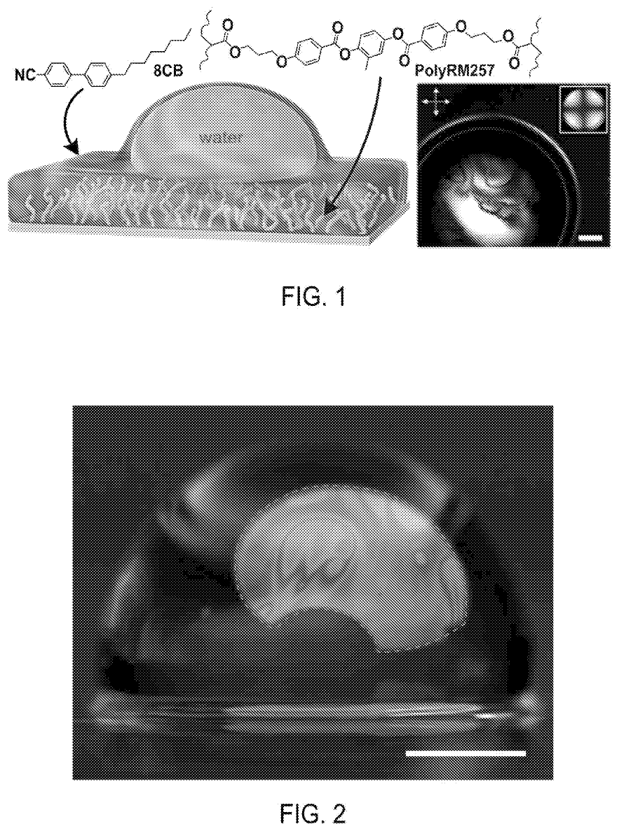

A 1 μL water droplet was deposited on a 130 μm-thick 8CB lubricating film-coated 8CB-swelled polyRM257 nanoporous structure on a DMOAP-functionalized glass slide at 35° C. (e.g., nematic). When observed under polarized light microscopy, the nematic 8CB film was dark in air and turned bright when it came in contact with a water droplet (1 μL), as shown in FIG. 1. This transition is consistent with different surface anchoring of nematic 8CB at the air-liquid crystal interface (perpendicular orientation) and water-liquid crystal interface (parallel orientation) (Kiernan M et al. Soft Matter Physics: An Introduction (Springer, 2003)). Additionally, inertia-driven droplet oleoplaning was observed at the nematic 8CB surface, which indicates that the porous polyRM257 substrate stabilized the nematic 8CB film and thus prevented water droplets from becoming pinned to the surface. This design principle is general and can be applied to a range of thermotropic liquid crystals such as 5CB and E7. Further, a thin 8CB wrapping layer existed around the water droplet, which can slow the evaporation of water droplets at liquid crystal surfaces (FIG. 2).

Next, the behavior of water droplets at the surface of 8CB in the smectic A phase, where the constituent molecules have both long-range orientational order and unidirectional positional order (e.g., liquid crystal molecules are arranged in rows perpendicular to the surface), was investigated. A 1 μL water droplet was deposited on a 130 μm-thick 8CB lubricating film-coated 8CB-swelled polyRM257 nanoporous structure on a DMOAP-functionalized glass slide at 25° C. (e.g., smectic). In contrast to the surface behavior when 8CB was in the nematic phase, focal conic domain arrays (Gim M J et al. Nat. Commun. 2017, 8, 15453; Honglawan A et al. Proc. Natl. Acad. Sci. U.S.A. 2013, 110, 34-39) formed at the smectic A 8CB-water interface, and water droplets became severely pinned to the surface.

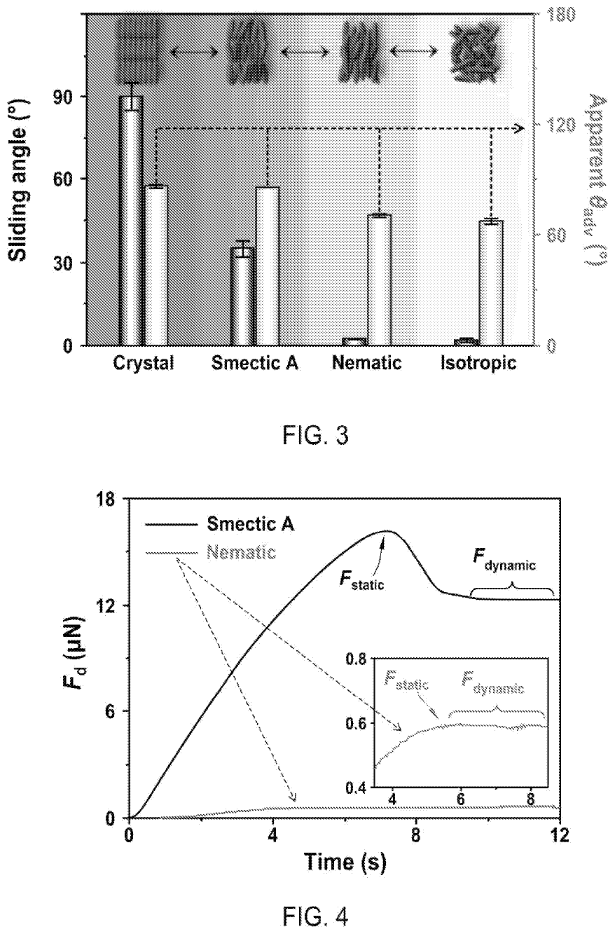

To investigate the liquid crystal mesophase-dependent sliding angles of water droplets, 3 μL water droplets were deposited on a 130 μm-thick 8CB lubricating film-coated 8CB-swelled polyRM257 nanoporous structure on a DMOAP-functionalized glass slide, which was then tilted at different angles and the angle at which the water droplets began to slide was measured with a contact angle goniometer. As summarized in FIG. 3, the apparent advancing contact angles (Oath) of a sessile water droplet on the 8CB surface are similar over a wide temperature range (5-50° C.), whereas the sliding angle of a 3 μL water droplet decreases from being completely pinned in the crystalline phase to −35° in the smectic A phase, down to −2° for both nematic and isotropic phases of 8CB.

Water droplets need to overcome a static pinning force (F_{static}) to start moving against dynamic friction (F_{dynamic}) at a surface (Gao N et al. Nat. Phys. 2017, 14, 191-196). To provide insights into the mechanism by which the liquid crystal mesophase affects droplet mobility at liquid crystal surfaces, the dissipative force (Fa) acting on a moving droplet was measured using a customized cantilever force sensor. For example, the liquid crystal mesophase-dependent friction behavior was examined by depositing 34 water droplets on a 130 μm-thick 8CB lubricating film-coated 8CB-swelled polyRM257 nanoporous structure on a DMOAP-functionalized glass slide. The temperature was set to 25 and 35° C. for smectic A and nematic phases, respectively. The substrate was rotated such that the tangential speed of the droplet was 0.5 mm/s. The k of capillary tubes for nematic and smectic A surface were 17.3 and 23.3 mN/m, respectively. As shown in FIG. 4, water droplets were highly pinned at the smectic A 8CB surface (F_{static}=16.2 μN) with F_{static}>F_{dynamic}, which is characteristic of the stick-slip mode (Gao N et al. Nat. Phys. 2017, 14, 191-196). Secondly, water droplets at the nematic 8CB surface exhibited a small F_{static} of 0.6 μN with a smooth transition from static to dynamic (F_{static} F_{dynamic}), which is consistent with the pinning-free slippery mode (Smith J D et al. Soft Matter 2013, 9, 1772-1780; Daniel D et al. Nat. Phys. 2017, 13, 1020-1025). Additionally, signatures of the slippery mode and stick-slip behavior was observed when the 8CB surface was isotropic (no intrinsic order) and crystalline (three dimensional positional order and long-range orientational order), respectively, which is strongly analogous to the behavior of isotropic materials (e.g., ordinary liquid and amorphous solid state of wax, respectively) (Yao X et al. Angew. Chem. Int. Ed. 2015, 54, 8975-8979). The above observations of liquid crystal mesophase-dependent surface slipperiness lead to the conclusion that positional order, rather than orientational order, dictates the frictional behavior of the liquid crystal surface.

Previous studies have demonstrated that the F_{dynamic} of droplets moving across isotropic oil-based SLIPS can be predicted by the Landau-Levich-Derjaguin (LLD) theory (Daniel D et al. Nat. Phys. 2017, 13, 1020-1025):

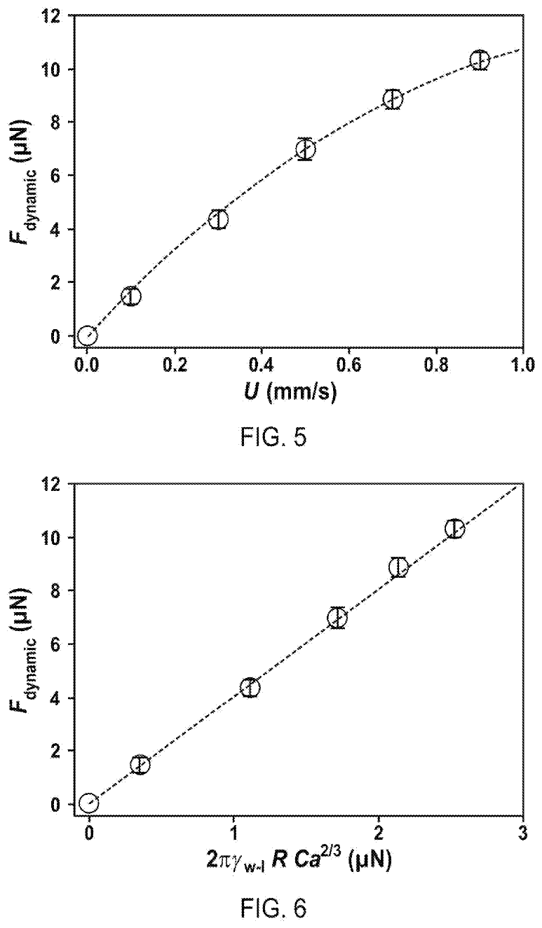

F_{dynamic}≈2πγ_{w-1}R Ca^{2/3} (3)

in which γ_{w-1} is the water-lubricant interfacial tension, R is the base radius of the droplet, and Ca is the capillary number, which is proportional to the speed at which the droplet moves across the surface (U). Herein, the question of whether nematic liquid crystal surfaces obey the LLD behavior was explored. As shown in FIG. 5, F_{dynamic} increases as U increases. This can be attributed to an increase in the thickness of the lubricating nematic 8CB layer, which in turn increases the viscous dissipation to water droplet motion. Importantly, the experimentally measured F_{dynamic} yielded a linear relationship with F_{dynamic} calculated using equation (3) (FIG. 6), suggesting that the behavior of water droplets on nematic liquid crystal surfaces follows the LLD law, just as SLIPS with isotropic lubricants. The line best fits the data with a viscosity of ˜20 cP, indicating degenerate planar anchoring of nematic 8CB at the water droplet surface (Chmielewski A G. Mol. Cryst. Liq. Cryst. 1986, 132, 339-352).

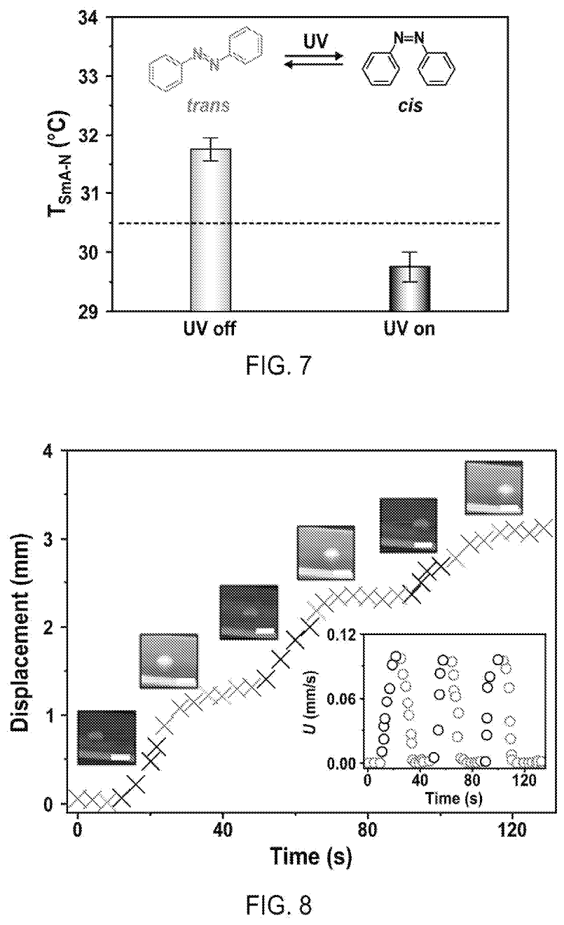

Optical manipulation of droplet mobility on liquid crystal surfaces. Past studies have shown that UV-induced cis-trans isomerization of doped azobenzene disrupts liquid crystal molecular order (Bukusoglu E et al. Annu. Rev. Chem. Biomol. Eng. 2016, 7, 163-196). To remotely manipulate the motion of water droplets at 8CB surfaces, azobenzene was added to 8CB at 1 wt % concentration, which resulted in a −2° C. shift in the smectic A-nematic (SmA-N) phase transition temperature upon exposure to UV radiation (FIG. 7). When the UV exposure was alternated on and off, the azobenzene-doped 8CB phase was switched repeatedly between smectic A (stick-slip mode) and nematic (slippery mode) phases, which caused droplets to repeatedly slide and become pinned on a tilted 8CB surface, as shown in FIG. 8.

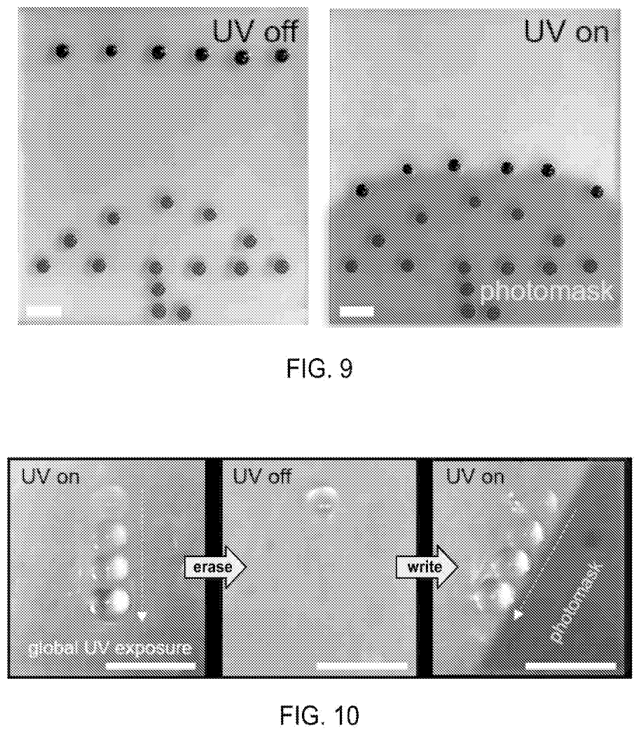

Next, patterning the UV radiation to program the positioning of water droplets at liquid crystal surfaces was investigated. As shown in FIG. 9, water droplets at portions of the liquid crystal surface covered with an aluminum foil photomask (which was not exposed to UV and hence remained in the smectic A phase) remained stationary, whereas water droplets on the UV-exposed liquid crystal surface (which transitioned to nematic phase) slid to the edge of the region covered by the aluminum foil. Furthermore, it was demonstrated that the trajectory of water droplets could be tuned by selective exposure of UV radiation. FIG. 10 reveals that the UV radiation pattern could be reversibly written and erased to guide the path of water droplets sliding down an inclined 8CB surface, which deviated from the trajectory droplets would normally follow due to gravity. These results demonstrate that the manipulation of the positional order of liquid crystals offers a multitude of possibilities that enable interfacial transport of liquid droplets in a programmable manner.

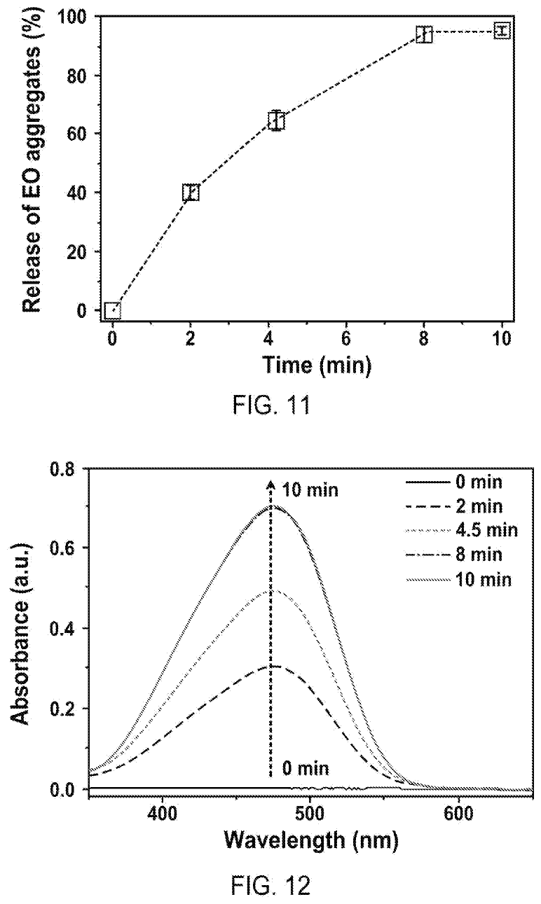

Activated cargo release from liquid crystal-infused porous surfaces. Recent studies have reported that thermally-triggered nematic-isotropic (N-I) transitions can activate the release of cargo droplets trapped within a flat liquid crystal film or liquid crystal droplets submerged in water (Kim Y K et al. Nature 2018, 557, 539-544; Guo J K et al. Adv. Sci. 2019, 6, 1900785), though the release of cargo to water droplets residing on liquid crystal surfaces has not been demonstrated to date. Herein, the activated release of liquid crystal-entrapped cargos to water droplets deposited on liquid crystal-infused porous surfaces was explored. Based on past observations of size-dependent ordering of confined liquid crystals (Yamamoto J et al. Nature 2001, 409, 321-325), two methods were used to load different sized cargos in the liquid crystal surface. First, the water-soluble dye ethyl orange (EO) was loaded in 8CB by using ethanol as a co-solvent, which was subsequently evaporated to form nano-sized ethyl orange aggregates within the bulk of 8CB. As shown in FIG. 11 and FIG. 12, ethyl orange was automatically released to water droplets placed on the surface of ethyl orange-dispersed 8CB.

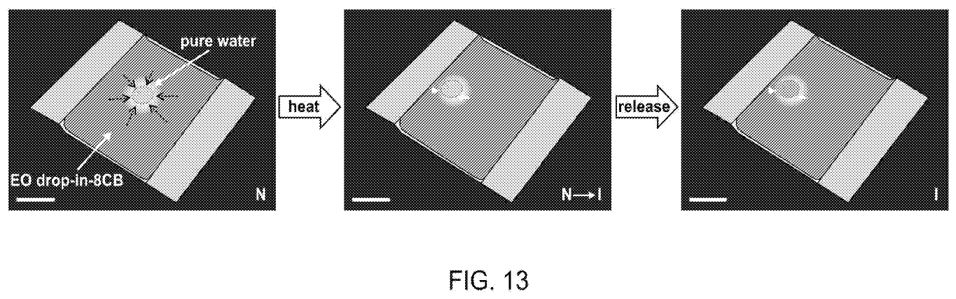

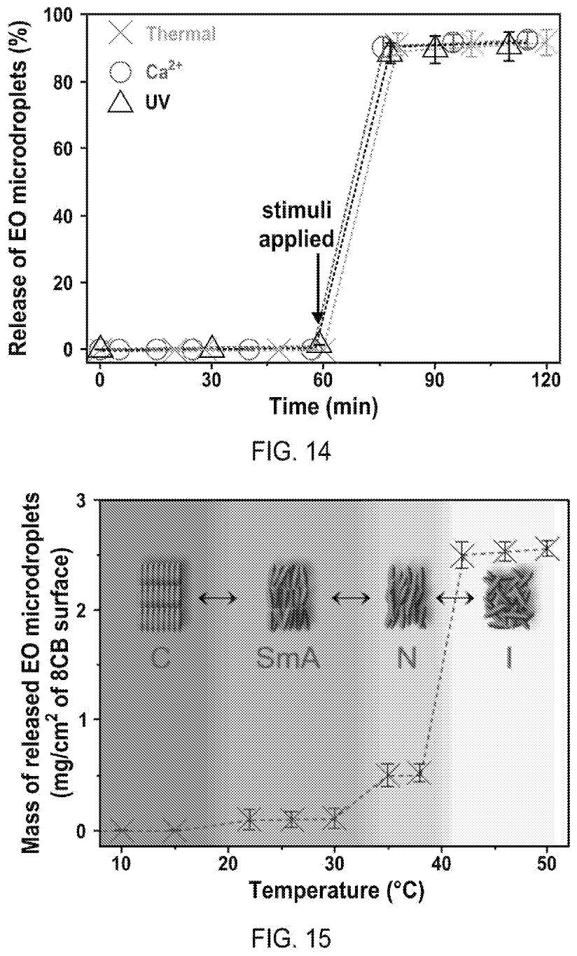

In the second method, an inverse emulsion of ethyl orange aqueous microdroplet-in-8CB was coated onto the 8CB-swelled porous polyRM257 substrate. The ethyl orange microdroplets initially concentrated in the wetting ridge around the water droplet, but did not enter the water droplet. However, upon the nematic-isotropic transition, more than 90% of the encapsulated ethyl orange microdroplets were continuously released into the water droplet, as shown in FIG. 13 and FIG. 14. Besides ethyl orange, it was observed that this phenomenon is generalizable to a range of water-soluble dyes such as Rhodamine B (RhB), methyl orange, and methylene blue.

The above observations of loading method-dependent release profiles on liquid crystal surfaces can be attributed to the size-dependent liquid crystal ordering around the guest cargo, which is determined by a competition between the elastic energy (KR_{cargo}, where K is the Frank elastic constant and R_{cargo} is the radius of the cargo microdroplet) and surface anchoring energy (WR_{cargo}², where W is the surface anchoring strength) (Kiernan M et al. Soft Matter Physics: An Introduction (Springer, 2003)). Specifically, the local mesogenic orientation around microcargos (R_{cargo}>K/W˜1 μm) prevents their automatic release from the liquid crystal surface, whereas nano-sized cargos with R_{cargo}<K/W that cause no orientational elastic distortion automatically release.

It was also observed that release of ethyl orange microdroplets to water droplets could be activated by either UV radiation or presence of Ca²⁺, as shown in FIG. 14. Finally, in addition to the nematic-isotropic transition, the other two first-order phase transitions of 8CB, i.e., crystal-smectic A and smectic A-nematic, were also measured to activate the release (FIG. 15). These observations lead to the conclusion that orientational order (rather than positional order) plays an essential role in cargo release processes.

Thermodynamic model for activated cargo release at liquid crystal surface A recent study of a flat liquid crystal film submerged in bulk water demonstrated that orientational elasticity at a nematic-isotropic biphasic interface triggers a pulsatile cargo release, with no further release of cargo microdroplets once the liquid crystal film has fully transitioned to isotropic (Kim Y K et al. Nature 2018, 557, 539-544). In the system used herein, it was reasoned that the capillary force induced by the curved capillary menisci (e.g., the wetting ridges) surrounding the water droplets concentrates the cargo microdroplets in the wetting ridge, and the capillary force alone is unable overcome the liquid crystal elastic barrier in the nematic phase. To provide insight into the role of the capillary force, the classic Derjaguin-Landau-Verwey-Overbeek (DLVO) model was modified to describe the essential change in the process of cargo release. The van der Waals force (F_{vdW}), capillary force (F_{cap}), electric double layer force (F_{edl}), and liquid crystal elastic force (Fₑₗ) were combined into a thermodynamic model to describe the essential behaviors of millimeter-sized water droplets interacting with liquid crystal surfaces during the process of activated cargo release. The net force (Fₙₑₜ) acting on the cargo microdroplet can be written as (see Supplementary Materials for details):

$${F}_{net} = {F}_{vdW} + {F}_{cap} + {F}_{edl} + {F}_{el} = - \frac{{A}_{H} {R}_{cargo}}{6 {x}^{2}} - \frac{2 {γ}_{LC} πξ {R}_{cargo}^{3}}{{( R + x )}^{2}} + 64 π {ε}_{0} {{ε}_{LC} ( {k}_{B} T / e )}^{2} tanh ( ze {ψ}_{cargo} / 4 {k}_{B} T ) tanh ( ze {ψ}_{drop} / 4 {k}_{B} T ) κ {R}_{cargo} {e}^{- κ x} + \frac{{α}^{2} β π {KR}_{cargo}^{4}}{{( {R}_{cargo} + x )}^{4}} \qquad ( 4 )$$

in which A_{H} is the Hamaker constant for the air-liquid crystal-water interface, R_{cargo} is the radius of the cargo microdroplet, x is the surface-to-surface distance between the cargo microdroplet and millimeter-sized water droplet, ξ is a coefficient to estimate the average hydrostatic pressure acting on each hemisphere, ε₀ is the vacuum permittivity, ε_{LC} is the relative permittivity of the liquid crystal, k_{B} is the Boltzmann constant, T is the temperature, z is the valence number of the dominant aqueous ionic species, e is the elementary charge, κ⁻¹ is the Debye length, and ψ_{cargo} and ψ_{drop} are the zeta potentials of cargo microdroplet and millimeter-sized water droplet, respectively. α and β denote constants related to liquid crystal, and K denotes the Frank elastic constant of liquid crystal.

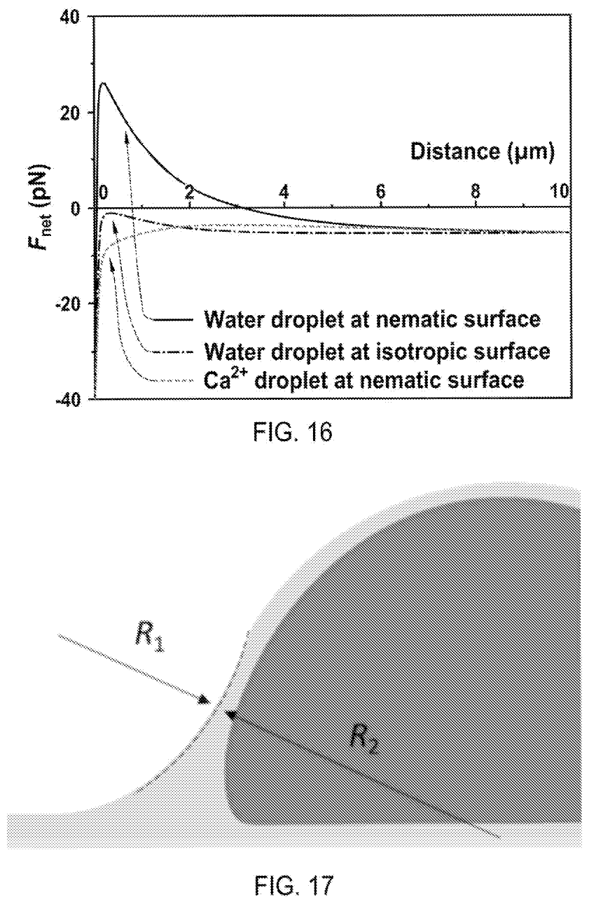

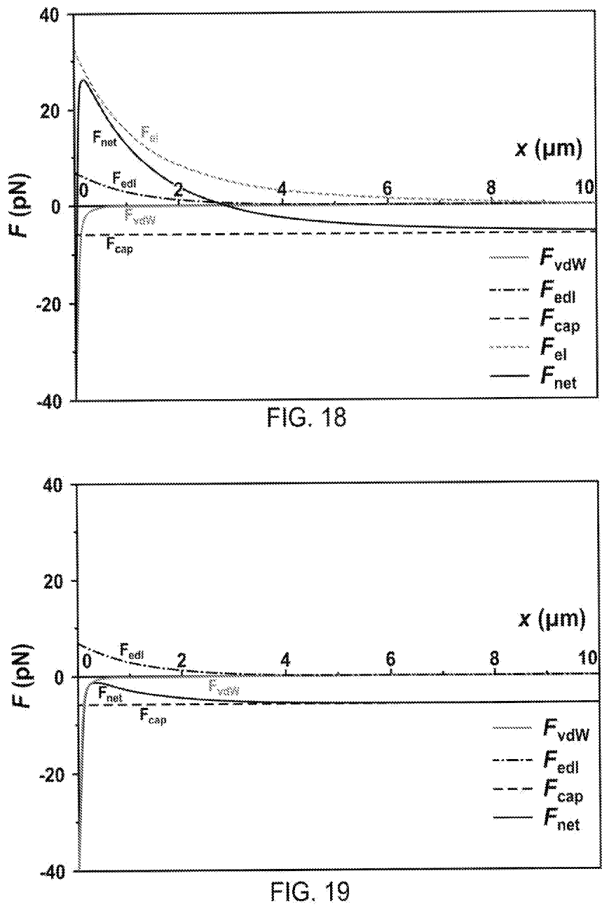

First, equation (4) was used to calculate Fₙₑₜ of cargo microdroplets encapsulated in a nematic liquid crystal surface with a deposited millimeter-sized droplet of pure water. The black curve in FIG. 16 shows Fₙₑₜ plotted alongside the individual forces from equation (4). The repulsive Fₑₗ and F_{edl} lead to a kinetic barrier (˜25 pN) that prevents ejection of cargo microdroplets (FIG. 18), which is consistent with the experimental observation that cargo microdroplets did not release into water droplets at a nematic liquid crystal surface.

Second, equation (4) was used to calculate Fₙₑₜ of cargo microdroplets (5 mM SDS aqueous solution of ethyl orange) encapsulated in an isotropic liquid crystal surface with a millimeter-sized droplet of pure water deposited on the surface. Fₑₗ=0 when the liquid crystal is in the isotropic phase. As shown in the red curve in FIG. 16 and FIG. 19, the absence of the repulsive Fₑₗ allows Fₙₑₜ to become negative (attractive), thus overcoming the repulsive F_{edl} and releasing the cargo into the water droplet. This result confirms that the nematic-isotropic transition induced by either heat or UV can activate the release of cargo microdroplets at the isotropic liquid crystal surface, which lead to the conclusion that the orientational order of the liquid crystal plays an essential role in the process of cargo release.

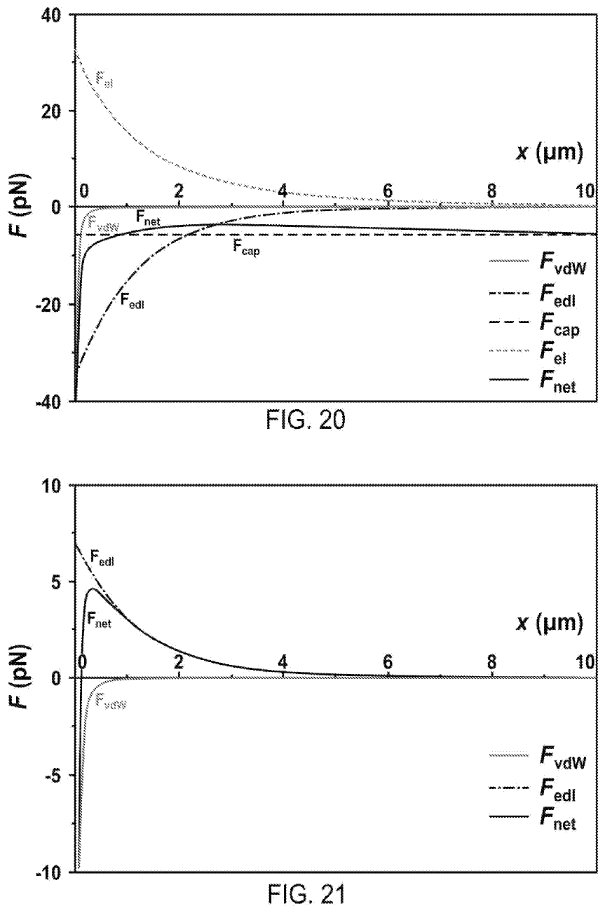

Third, equation (4) was used to calculate Fₙₑₜ of cargo microdroplets (5 mM SDS aqueous solutions of ethyl orange) encapsulated in nematic liquid crystal surfaces with millimeter-sized droplets of 10 mM Ca²⁺ deposited on the surface. The presence of cations changes the sign of ti/drop from positive to negative, resulting in an attractive F_{edl} acting on the cargo microdroplet. As shown in the blue curve in FIG. 16 and FIG. 20, this causes Fₙₑₜ to become negative for all x, showing that the attractive F_{edl}, F_{vdW}, and F_{cap} can overcome the repulsive Fₑₗ barrier. This result agrees with the experimental observation of activated release of cargo microdroplets by the presence of Ca²⁺. In summary, these results demonstrate that the thermodynamic model can qualitatively describe the essential behaviors in the process of cargo release at liquid crystal surfaces.

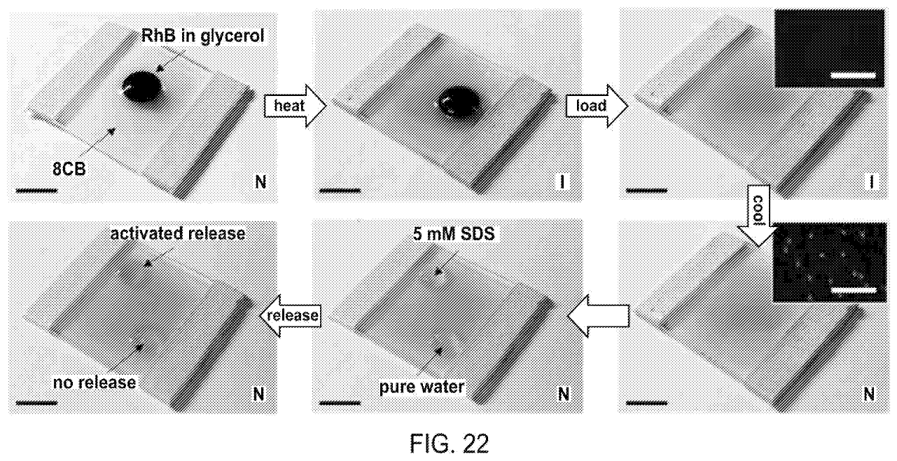

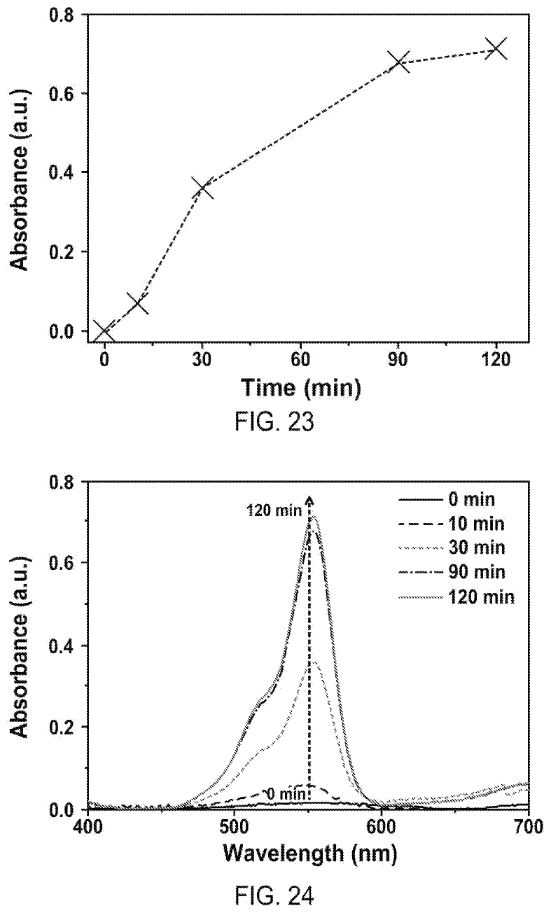

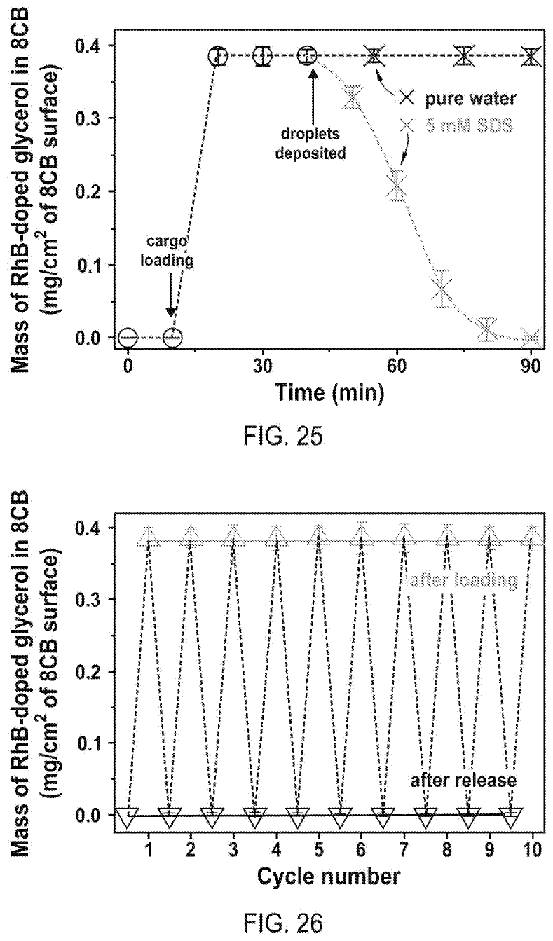

Reversible loading and release by liquid crystal-infused surfaces. Conventional drug delivery systems such as layer-by-layer polyelectrolyte assembly (Costa R R et al. Chem. Soc. Rev. 2014, 43, 3453-3479) have been demonstrated to program the cargo release profile, however, reversible, in-situ cargo loading and release remains challenging. Building from prior findings of mesophase-dependent solubility of glycerol in bulk liquid crystal (Nazarenko V G et al. Phys. Rev. Lett. 2001, 87, 075504), reversible switching of liquid crystal orientational order was taken advantage of to achieve reversible loading and release of cargos. As indicated in FIG. 22, Rhodamine B-doped glycerol that was initially dispersed in isotropic 8CB could automatically release to pure water droplets (FIG. 23 and FIG. 24), whereas Rhodamine B-doped glycerol microdroplets dispersed in nematic 8CB films could only be released with the aid of an anionic surfactant (FIG. 25). This cargo size-dependent loading and release behavior could be repeated more than ten consecutive times without any damage to the 8CB surface, as shown in FIG. 26. This is the first experimental demonstration of reversible loading and release of cargo on liquid crystal surfaces via switching mesogenic orientational order.

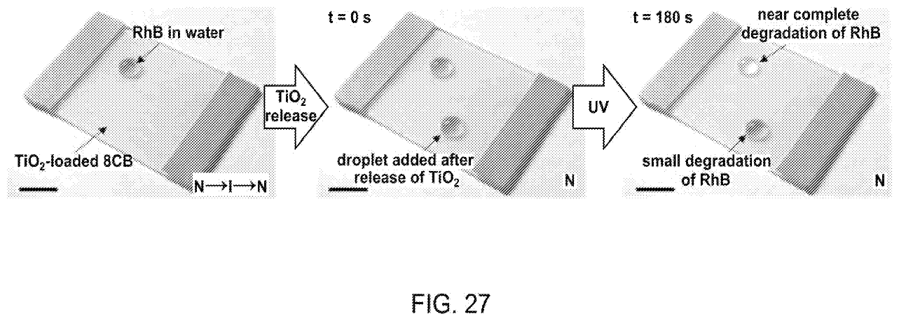

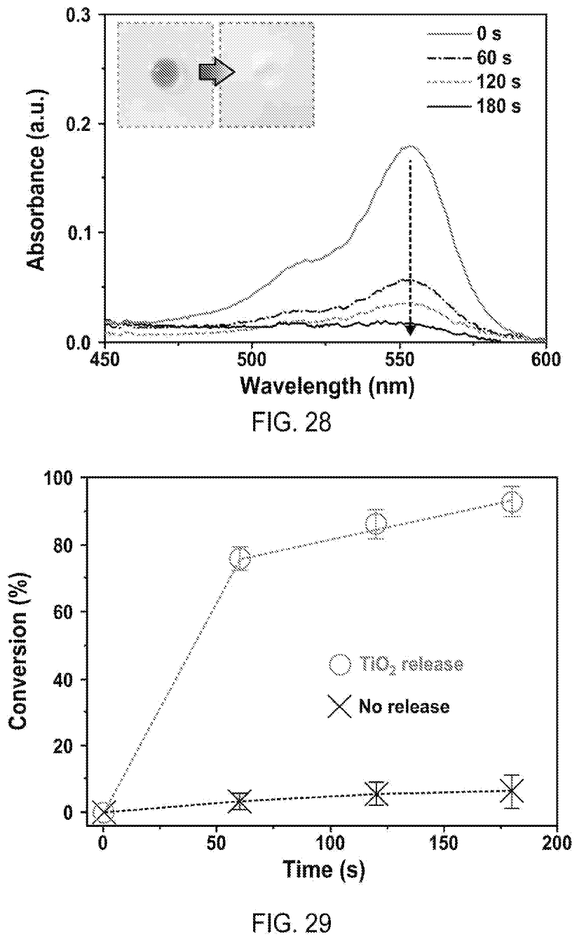

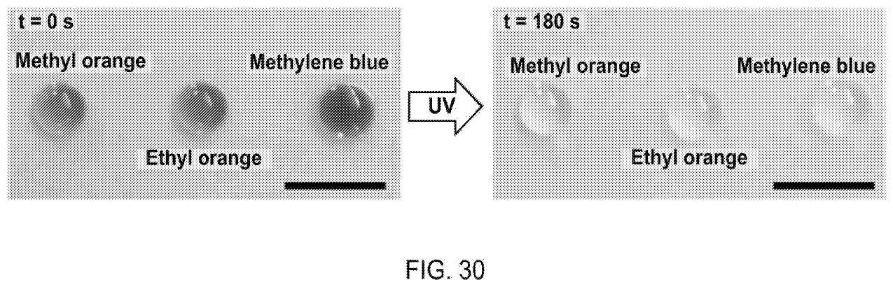

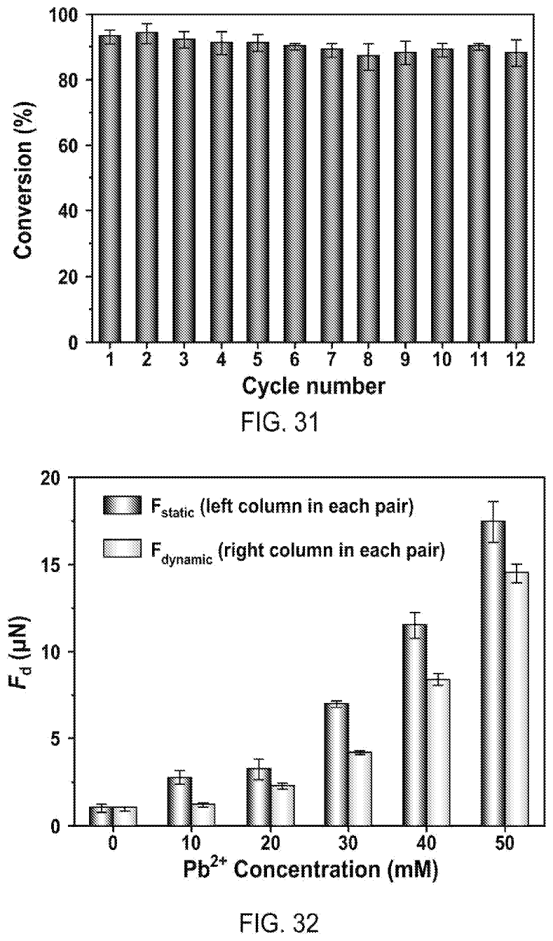

Removal of organics and heavy metal ions from water using liquid crystal surfaces. Over the past few decades, the monitoring and treatment of water pollution such as organics and heavy metal ions have become increasingly important for social and environmental sustainability. Herein, creation of liquid crystal-infused surfaces with complex functionalities for wastewater diagnosis and treatment was sought. First, liquid crystal surfaces were designed with tunable reactivity that can be used to degrade toxic organic substances in water. As shown in FIG. 27-FIG. 29, after the thermally-triggered release of TiO₂ from 8CB into Rhodamine B water droplets placed on the 8CB surface, aqueous Rhodamine B droplets (which were initially pink) turned colorless under UV exposure as the Rhodamine B dye was degraded by TiO₂-mediated photocatalytical chemistry (Liu K et al. Chem. Rev. 2014, 114, 10044-10094), whereas UV radiation in the absence of TiO₂ nanoparticles did not lead to considerable degradation of Rhodamine B. Moreover, this 8CB surface with tunable photocatalytical ability is generalizable to other water-soluble dyes (FIG. 30), and exhibited good stability and reusability, with the photocatalytic performance remaining highly efficient over 12 consecutive cycles (FIG. 31).

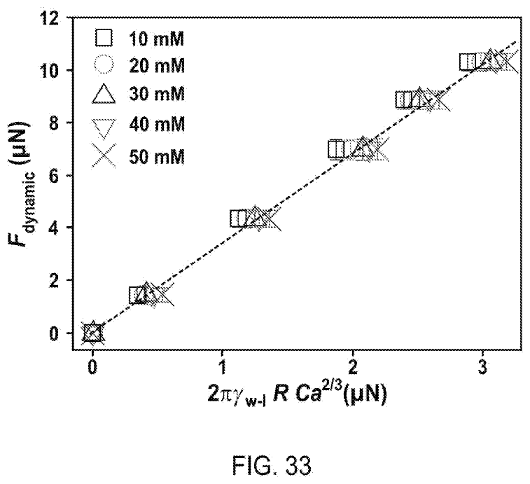

Next, the possibility of using liquid crystal surfaces to capture heavy metal ions that have been shown to cause physical, muscular, and neurological diseases (Fu F et al. J. Environ. Manage. 2011, 92, 407-418) was explored. First, F_{d} acting on aqueous droplets at nematic 8CB surfaces was quantified as a function of Pb²⁺ concentration. FIG. 32 shows that F_{static} increased by a factor of ˜10 when Pb²⁺ concentration increased from 0 to 50 mM, which occurred due to the increase of contact line pinning of Pb′ droplets at nematic 8CB surfaces (see Supplementary Materials). An increase in F_{dynamic} was also measured with Pb²⁺ concentration, which occurred due to an increase in the viscous dissipative force predicted by LLD theory (see FIG. 33 and Supplementary Materials). These results indicate that measuring F_{d} at liquid crystal surfaces provides a new way to quantify the concentration of cations in water droplets.

Finally, precipitation of heavy metal ions from water droplets was sought by doping the 8CB surface with aqueous droplets of sulfide anions S²⁻. A 3 μL pure water droplet and 3 μL aqueous droplets of 10 mM Pb²⁺ were deposited onto an 8CB film loaded with 20 wt % of 3 mM SDS and 40 mM Na₂S. The cargo-loaded 8CB film was coated on a 8CB-swelled polyRM257 nanoporous structure on a DMOAP-functionalized glass slide. The surface was inclined from the horizontal position at 3°. The temperature was maintained at 35° C.

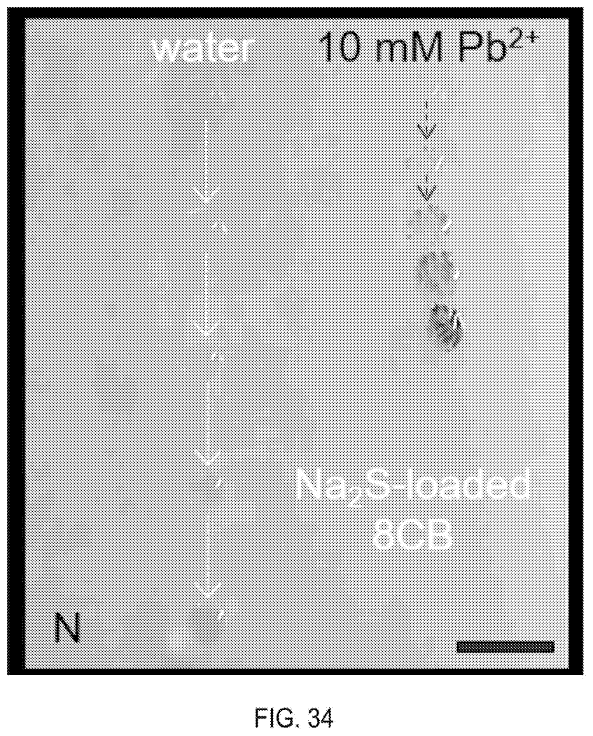

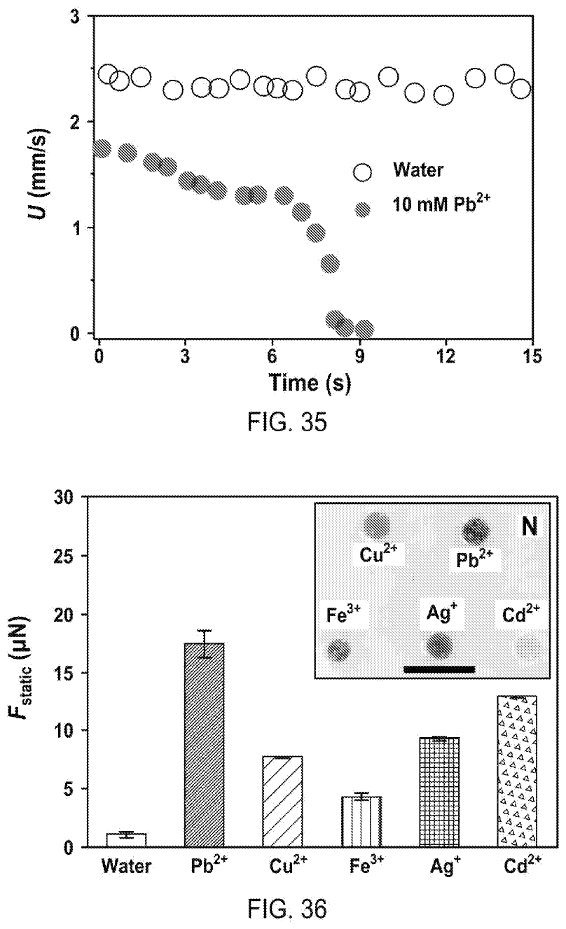

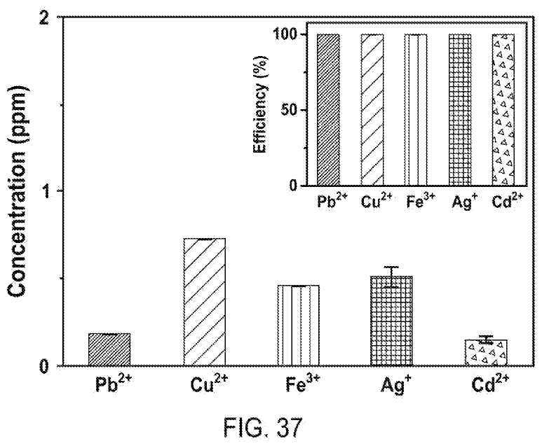

As shown in FIG. 34 and FIG. 35, when 10 mM Pb²⁺ droplets were in contact with 8CB film, S²⁻ was released and reacted with Pb²⁺ to form a black PbS precipitate, which induced the slowing and eventual pinning of Pb²⁺-containing droplets, whereas droplets of pure water continued to slide off the surface without slowing down. Besides Pb²⁺, this approach can be generalized for a range of heavy metal ions including Cd²⁺, Fe³⁺, Ag⁺ and Cu²⁺ (FIG. 36), and the concentration of leftover heavy metal ions in aqueous droplets was measured to be less than 1 ppm (FIG. 37). This is the first experimental evidence that liquid crystal surfaces can self-sense and self-clean pollutants in water with ultrahigh removal efficiency.

Discussion. The data above demonstrates that the droplet mobility and cargo release can be tuned by the molecular order of constituent mesogens of the liquid crystal surface. Three comments regarding the characteristic behaviors of the liquid crystal surface using FIG. 16 and FIG. 17-FIG. 21 are noted. First, the long-distance F_{cap} caused by curved capillary meniscus of liquid crystal wetting ridge provides a driving force to transport the cargo microdroplets to the wetting ridge and activate the subsequent cargo release, which is consistent with experimental observations (FIG. 13 and FIG. 14). This analysis lead to a hypothesis that the absence of F cap in a past study of flat liquid crystal surfaces was responsible for the pulsatile release of cargo microdroplets driven by nematic-isotropic phase transition, without further continuous release in isotropic phase (Kim Y K et al. Nature 2018, 557, 539-544). The thermodynamic model predicts that when the liquid crystal film is immersed under bulk pure water, the absence of the curved wetting ridge (F_{cap}=0) leads to a purely repulsive force barrier that prevents the release of cargo at flat isotropic surfaces (FIG. 21), which is in qualitative agreement with the past study of flat liquid crystal film under bulk water (Kim Y K et al. Nature 2018, 557, 539-544).

Secondly, moving cargo microdroplets also experience a Stokes drag force (Fₛ=−6πην_{cargo}R_{cargo}, where η is the dynamic viscosity of the liquid crystal and ν_{cargo} is the velocity of the cargo microdroplet), which acts opposite to the direction of the velocity of the cargo microdroplets. The effect of Fₛ was not included in equation (4) since only thermodynamics (rather than kinetics) were considered in the thermodynamic model.

Third, while the thermodynamic calculation qualitatively supports experimental observations, there were some ambiguities in selecting values for constants during the calculations. Therefore, the calculations should not be expected to provide precise quantitative agreement with the experiments. Experimentally measured constants, additional theory, and computational studies are needed to develop an exact quantitative thermodynamic model. Future works could also incorporate Fₛ and other relevant kinetic parameters to establish time scales for cargo release to provide additional control in systems utilizing liquid crystal surfaces.

In conclusion, the results demonstrate that liquid crystal positional order dominates surface friction behavior, whereas mesogenic orientational order plays a pivotal role in the process of cargo loading and release. More broadly, it is believed that these results hint at new interfacial designs of liquid crystal materials that expands the potential utility of conventional isotropic lubricant-based SLIPS systems into the realm of open surface microfluidics, smart surface reactors, and water treatment. Recent research exploring active liquid crystals (Duclos G et al. Science 2020, 367, 1120-1124; Doostmohammadi A et al. Nat. Commun. 2018, 9, 3246) hints at another promising direction that explores how dynamics of active matter encapsulated inside thermotropic liquid crystal surfaces influence the behavior of water droplets deposited on the top. Recent studies have also reported the use of biocompatible liquid crystal materials in tissue regeneration (Gao Y et al. ACS Macro Lett. 2015, 5, 4-9) and alignment of human cells (Turiv T et al. Sci. Adv. 2020, 6, eaaz6485) and bacteria (Peng C et al. Science 2016, 354, 882-885), which suggests that the liquid crystal surfaces described herein can be utilized in in vivo drug delivery. Future efforts will seek to explore droplet mobility and activated release at the surface of other liquid crystal mesophases, including cholesteric phase and blue phases. Finally, based on recent study of dynamics of multiple droplets on isotropic surfaces (Cira N J et al. Nature 2015, 519, 446-450), intrinsic molecular order embedded in liquid crystal surfaces can offer new avenues to manipulate multi-droplet motion and interactions.

Materials and Methods

Materials. The following liquid crystal monomers were purchased from Jiangsu Hecheng Advanced Materials Co., Ltd: 4′-pentyl-cyanobiphenyl (5CB), 4′-octyl-4-biphenylcarbonitrile (8CB), E7, and 1,4-Bis-[4-(3-acryloyloxypropyloxy)benzoyloxy]-2-methylbenzene (RM257). The following chemical compounds were purchased from Sigma-Aldrich: water-soluble dyes (ethyl orange (EO), Rhodamine B (RhB), methyl orange and methylene blue), anionic surfactant sodium dodecyl sulfate (SDS), nonionic surfactant polyoxyethylene (10) oleyl ether (Brij 97), dimethyloctadecyl[3-(trimethixysilyl)propyl]ammonium chloride (DMOAP, 42 wt % in methanol), photoinitiator 2,2-dimethoxy-2-phenylacetophenone (DMPAP), salts (CaCl₂), Na₂S, AgNO₃, PbNO₃, CuSO₄, CdCl₂, and FeCl₃), poly(pyromellitic dianhydride-co-4,4′-oxydianiline) amic acid (PAA), and 1-methyl-2-pyrrolidinone. Titanium dioxide (TiO₂) nanoparticles averaging 100 nm in diameter was purchased from Alfa Aesar. Glycerol and azobenzene were purchased from Fisher Scientific. Anhydrous ethanol was purchased from Decon Labs Inc. Transmission electron microscopy (TEM) grids (G75-Au; 3.05 mm-in-diameter) were purchased from Electron Microscopy Sciences. Water used in all experiments was purified using a Milli-Q water purification system (Simplicity C9210). Unless stated otherwise, purchased chemicals were used as received without further modification or purification.

Preparation of DMOAP-functionalized glass slides. First, glass slides were rinsed with water and ethanol and dried under a stream of nitrogen gas. Then, the cleaned glass slides were placed in a 1% v/v DMOAP water solution (120 mL) for 15 min. Afterwards, the glass slides were washed first with water and then with ethanol to remove unreacted DMOAP molecules. Afterwards, the DMOAP-functionalized glass slides were dried using nitrogen gas. These slides were stored in a dark room at ambient pressure and temperature to prevent light from damaging the DMOAP coating.

Preparation of polyimide-coated glass slides. A Laurell WS-650Mz-23NPPB spin processor was used to spin-coat a mixture of PAA (10% v/v) and 1-methyl-2-pyrrolidinone (90% v/v) on a cleaned glass slide at 4,000 rpm for 2 min. This glass slide was then heated to 350° C. for 3 h to coat the glass with polyimide. Finally, a velvet cloth was used to rub the surface of the polyimide unidirectionally 60 times.

Preparation of liquid crystal-filled TEM grids. A 20 μm-thick gold TEM grid was placed on a DMOAP-functionalized glass slide. Next, each mesh of the TEM grid was filled with 5CB via syringe to obtain an approximately flat 5CB interface.

Preparation of liquid crystal-infused porous surfaces. To make the liquid crystal-infused porous surfaces, a liquid crystal mixture containing non-reactive liquid crystal mesogens (8CB, 90 wt %) and a reactive liquid crystal monomer (RM257, 10 wt %) was prepared. DMPAP was added to the mixture as a photoinitiator at 1 wt % based on the total mass of the liquid crystal. Next, 100 μL of the homogenous liquid crystal mixture was uniformly spread across a 2.5 cm×2.5 cm DMOAP-functionalized glass slide. Subsequently, the liquid crystal mixture-coated glass was placed under a UV lamp (Spectroline, EA-140; 365 nm) and exposed at 2.0 mW/cm 2 for 20 min at 35° C. to prepare a uniform −160 μm-thick 8CB-swelled porous structure. Finally, the same non-reactive liquid crystal mesogen (8CB; 80 μL) was drop-casted onto the 8CB-swelled polyRM257 nanoporous structure to form a 130 μm-thick 8CB lubricating film. The azobenzene-doped liquid crystal-infused porous structure was fabricated by using the same protocol as reported above for 8CB-infused porous surfaces but using a mixture of azobenzene-doped 8CB (1 wt % azobenzene) instead of pure 8CB. The optical appearance of the 8CB surface with deposited water droplets was recorded using an Olympus BX53 microscope equipped with polarizers.

Imaging of liquid crystal wrapping layer of water droplets on liquid crystal surfaces. The liquid crystal wrapping layer that encapsulated water droplets at nematic 8CB surfaces was imaged using a custom-made color interferometer. A 3 μL water droplet was illuminated using diffuse white LEDs, and the interference patterns were captured using a Canon digital single lens reflex (DSLR) camera.

Goniometer measurement. A KRUSS DSA 100 goniometer was used to measure contact angles (both advancing and receding) and sliding angles using the sessile drop method, and interfacial tensions using the pendant drop method, which calculates interfacial tensions or forces from the shape of a suspended droplet. During these measurements, the subject liquid was pushed through the needle slowly (5 μL/min) to minimize the effect of dynamic forces on the shape of the droplet. Images of the pendant droplet near departure were captured and analyzed using a drop shape analyzer to estimate interfacial forces. A Linkam PE120 Peltier hot stage was used to control the temperature of the liquid crystal surface during these measurements. Contact angles and sliding angles were measured using 3 μL water droplets that were deposited on the surface using the liquid dispensing feature of the goniometer. For sliding angle measurements, 3 μL water droplets were placed on the liquid crystal surface and tilted the surface at a rate of 1°/min using a built-in command on the goniometer. The angle of inclination at which a droplet began to slide was its sliding angle. The air-liquid crystal surface and water-liquid crystal interfacial tensions were measured using the pendant droplet method, and average values were calculated from approximately 10 measurements taken from 3 different droplets. For water-liquid crystal interfacial tension measurements, water was placed inside a quartz cell. The liquid crystal was placed in a syringe with a needle tip which was held under the surface of the water for the pendant drop method. A high-resolution camera captured images of these droplets, which were then used to calculate the surface/interfacial tensions using a built-in software.

F_{d} measurements by cantilever force sensor. F_{d} acting on moving aqueous droplets was measured using a custom-made cantilever consisting of an acrylate needle (whose tip was sealed with epoxy) with inner and outer diameters of 300 and 700 μm, respectively. The cantilever was fixed on the goniometer and a ThorLabs PRM1Z8 brushless DC motor rotated the substrate with an angular velocity of 0.1-5.0°/s. Droplets were adhered to the side of the acrylic needle 1-2 cm away from the center of rotation. This caused the needle to deflect from the equilibrium position when the stage rotated. This process was recorded using a camera on the goniometer, and the deflection of the capillary tube Δx was measured using the open-source software Tracker. F_{d} was calculated using Hooke's law as:

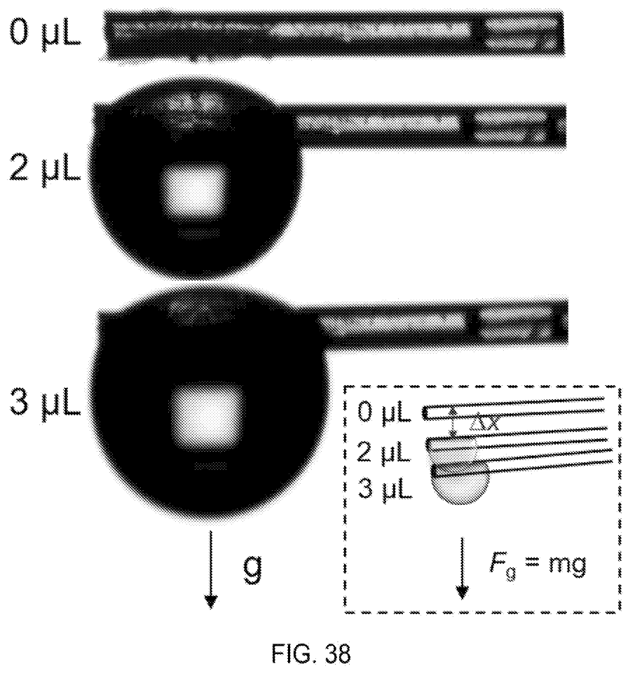

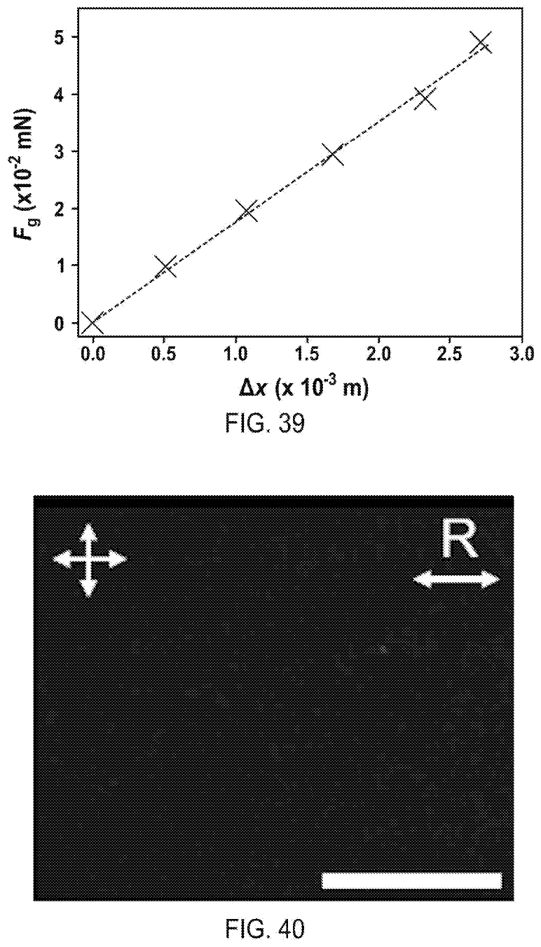

F_{d}=kΔx (5)

where k is the spring constant of the needle, which was measured by placing droplets of different volumes on the horizontally positioned capillary tube and measuring the deflection of the tip position in the z-direction (FIG. 38-FIG. 39). For F_{d} measurements on nematic and smectic A phases of 8CB, capillary tubes with k values of 17.3 and 23.3 mN/m, respectively, were used. This approach was sensitive enough to resolve changes in F_{d} as small as 0.1 μN.

Tuning mobility of water droplets on azobenzene-doped 8CB surfaces via UV radiation. First, the phase transition temperature of azobenzene-doped 8CB was determined using polarized light microscopy. The temperature was controlled by a Linkam PE120 Peltier hot stage, which allowed the temperature to be changed at a rate of 0.5° C./min. To examine the photo-responsive droplet mobility on azobenzene-doped liquid crystal surfaces, the azobenzene-doped 8CB surface was kept at 30.5° C. and the surface was tilted to a 3° inclination. Then, a 3 μL water droplet was placed on the 8CB surface, which was initially pinned to the surface. Next, the 8CB surface was periodically exposed to UV to tune the mobility of water droplets on 8CB surface.

Tunable positioning and trajectory of water droplets on azobenzene-doped 8CB surfaces through patterned UV radiation. The azobenzene-doped 8CB surface was kept at 30.5° C. An array of 3 μL water droplets was deposited on a 130 μm-thick 8CB film (doped with 1 wt % azobenzene)-coated 8CB-swelled polyRM257 nanoporous structure on a DMOAP-functionalized glass slide. More specifically, dark blue droplets (60 mM methylene blue) were placed at the top of the surface and acted as “rain”, while red droplets (60 mM Rhodamine B) were placed at the bottom of the surface in the pattern of an umbrella. The volume of each droplet was 3 μL. The surface was inclined from the horizontal position at 30°. Then, the red droplets were covered with an aluminum foil photomask and the entire liquid crystal surface was exposed to UV light (365 nm). After 3 min of continuous exposure, it was found that the uncovered blue water droplets (rain) had slid down the surface, while red droplets (which were masked with aluminum foil) had not moved.

Tunable trajectory of water droplets on azobenzene-doped 8CB surfaces through patterned UV radiation. An azobenzene-doped (1 wt %) 8CB surface was kept at 30.5° C. and a 3 μL droplet of water was placed upon it, which was initially pinned to the surface. Next, the entire liquid crystal surface was illuminated with UV light (365 nm). The water droplet slid, following a vertical trajectory due to the force of gravity. Second, UV radiation was stopped for 10 min and a new water droplet (3 μL) was placed on the surface. This droplet was initially pinned to the surface. Next, a region of the liquid crystal surface was covered with an aluminum photomask, which was positioned such that the interface between the exposed and unexposed regions followed a diagonal line relative to the direction of gravity (the dotted line in FIG. 10). Then, the surface was illuminated with UV light for 3 min, after which the surface was tilted at a 30° inclination. It was observed that the water droplet slid down the surface following the diagonal interface between the exposed and unexposed region.

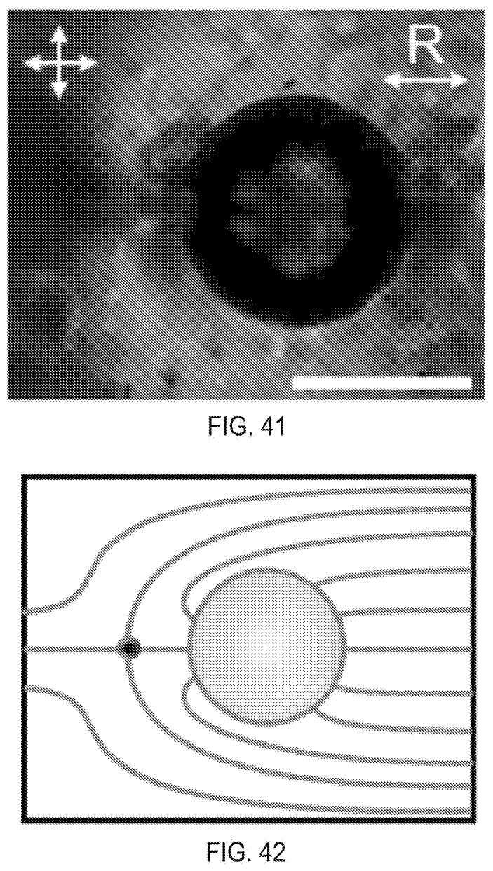

Methods of loading cargo into 8CB surfaces. Two different methods were used to load ethyl orange into 8CB surfaces. Co-solvent method: Ethanol was used as a co-solvent to prepare an ethyl orange/ethanol/8CB mixture, where the concentration of ethyl orange was 0.4 wt % based on the mass of 8CB. Subsequently, the ethanol was evaporated in a vacuum oven, leaving behind 8CB doped with nano-sized aggregates of ethyl orange. Finally, the obtained mixture was drop-casted onto an liquid crystal porous surface to develop a cargo-loaded 8CB-infused porous surface. Inverse emulsion method: 20 wt % of a solution containing 5 mM SDS and 1 mM ethyl orange and 80 wt % of 8CB at 35° C. was mixed to make a water-in-liquid crystal inverse emulsion. Next, this inverse emulsion was drop-casted onto a liquid crystal porous substrate. Finally, polarized light microscopy was used to image the ordering of nematic 8CB around the cargos prepared by the above two different methods in optical cells (FIG. 40-FIG. 42). The optical cells were 100 μm thick and were prepared by pairing two polyimide-coated glass slides.

Cargo loading and release determined by gravimetry. Cargo release at liquid crystal surfaces was quantified using gravimetry. Specifically, the mass of the 8CB-swelled polyRM257 nanoporous structures on a DMOAP-functionalized glass substrate (m₁) was first measured using a Mettler Toledo analytical balance. Next, cargo-loaded 8CB was drop-casted onto the obtained 8CB/polyRM257 surface and its mass (m₂) was recorded. The mass of loaded cargo was calculated as (m₂−m₁) y, in which y is the mass fraction of loaded cargo based on the mass of cargo-loaded 8CB. After activated release of cargos to the water droplets placed at the 8CB surface, the water droplets were removed and the mass of the 8CB surface (m₃) was measured. The mass of the released cargo was calculated as m₂−m₃, and the percentage of release of cargo loaded in 8CB surface was calculated as:

Percentage of cargo release=(m₂−m₃)/[(m₂−m₁)y]×100% (6)

In the experiments where reversible loading and release of glycerol at 8CB surfaces was investigated, glycerol was first loaded into an isotropic 8CB surface (see “Design of liquid crystal-infused surface for reversible in-situ loading and release of cargo”), the excess glycerol was removed, and the entire mass of the glycerol-loaded 8CB-infused porous surface (m₄) was measured. After the activated release of cargos to SDS aqueous droplets placed at nematic 8CB surface, the water droplets were removed and the new mass of the glycerol-loaded 8CB surface (m₅) was measured. The masses of the glycerol that were loaded into the isotropic 8CB surface and released from the nematic 8CB surface were calculated as m₄−m₂ and m₄−m₅, respectively.

UV-visible spectrophotometry measurements. The concentration of dyes in water droplets was determined using a Perkin Elmer Lambda 950 UV-visible spectrophotometer. After releasing the cargos into water droplets that had been placed on the 8CB surface, 5 μL aliquots were withdrawn from the droplet, placed into UV cuvettes, and diluted with 995 μL, of water. Then, the UV-visible absorption spectra ranging from 200 to 800 nm was measured.

Automatic release of cargo from 8CB surfaces. A 10 μL water droplet was placed onto a 2.5 cm×2.5 cm 8CB surface that was doped with nano-sized ethyl orange aggregates (0.4 wt %) using the co-solvent method described above. It was observed that the droplet turned orange, indicating that embedded cargo molecules were continuously released into the aqueous droplet.

Stimuli-activated release of cargo from 8CB surfaces. External stimuli (e.g., thermal, charge, and UV) were applied to trigger the release of loaded cargo. For these experiments, all 8CB surfaces were 2.5 cm×2.5 cm and doped with 20 wt % of aqueous droplets (containing 5 mM SDS and 1 mM ethyl orange) using the inverse emulsion approach described above. Thermally triggered release: A 10 μL water droplet was deposited on a nematic 8CB surface loaded with ethyl orange microdroplets at 35° C. Upon increasing the temperature to 45° C. (isotropic), the ethyl orange microdroplets, which were initially concentrated in the wetting rim of the water droplet, released into the water droplet. Charge-triggered release: 10 μL aqueous droplets of Ca²⁺ (10 mM) were deposited onto a nematic 8CB surface loaded with 20 wt % of aqueous droplets (containing 5 mM SDS and 1 mM ethyl orange) at 35° C. As a control, a droplet of pure water was also placed on the liquid crystal surface. The Ca²⁺ aqueous droplets turned orange while the droplet of pure water did not. UV-triggered release: A 5 mM SDS solution consisting of 1 mM ethyl orange with 1 wt % azobenzene-doped 8CB was mixed at 38° C. following the previously described protocol. Then, a droplet of water was placed onto this 8CB surface while maintaining the temperature at 38° C. The ethyl orange microdroplets released to the droplet after being exposed to UV light.

Design of liquid crystal-infused surfaces for reversible in-situ loading and release of cargo. 30 μL of a mixture of Rhodamine B-doped glycerol (20 mM Rhodamine B) was deposited on top of a 130 μm-thick 8CB film (doped with 1 wt % azobenzene)-coated 8CB-swelled polyRM257 nanoporous structure on a DMOAP-functionalized glass slide and the temperature was kept at 70° C. for 10 min. During this period, the Rhodamine B/glycerol mixture droplet was moved to different locations to spread the cargo molecules uniformly across the liquid crystal surface. Afterwards, the excess glycerol was removed using a Kimwipe. Next, the Rhodamine B/glycerol-loaded 8CB surface was cooled from 70° C. to 35° C. (isotropic-nematic transition), after which a 10 μL droplet of pure water and a 10 μL aqueous droplet of SDS (5 mM) were simultaneously placed on the surface. The cargo molecules were released only to the SDS aqueous droplet, while no release was observed for the pure water droplet. In addition, an Olympus IX73 fluorescent microscope with a 100 W mercury lamp was used to image the condensation of glycerol during isotropic-nematic transition.

Photocatalyzed degradation of aqueous organic contaminants by TiO₂-loaded liquid crystal surfaces. A 0.8 mM nonionic surfactant Brij 97 solution consisting of TiO₂ (1 wt % based on the Brij 97 solution) was mixed with 8CB (80 wt %). Then, 80 μL of the obtained mixture was drop-casted onto an 8CB-swelled polyRM257 nanoporous substrate to prepare a TiO₂-doped 8CB surface. Next, a 10 μL aqueous droplet of Rhodamine B (0.2 mM) was deposited onto this surface at 35° C., and the temperature was increased to 45° C. (isotropic) to release the loaded TiO₂ to the droplet. After 20 s, the surface was cooled down to 35° C. The Rhodamine B droplet was initially pink but turned colorless after 3 min of UV exposure. The same procedure was used to with other aqueous dyes (methyl orange, ethyl orange, methylene blue, all 0.6 mM) with similar results (FIG. 30).

Removal of heavy metal ions from water at 8CB surfaces. A 3 mM SDS solution consisting of Na₂S (40 mM) was mixed with 8CB (80 wt %). Afterwards, the obtained inverse emulsion mixture was applied onto the liquid crystal porous substrate. Next, 10 mM aqueous solutions of different heavy metal ions (Pb²⁺, Cd²⁺, Cu³⁺, Fe³⁺, and Ag⁺) were prepared, and 3 μL aqueous droplets of these solutions were placed onto the Na₂S-loaded 8CB surface, causing the heavy metal ions to react with S²⁻ anions and precipitate. To measure the remaining concentration of aqueous heavy metal ions, this procedure was repeated using 15 μL, aqueous droplets of different heavy metal ions (10 mM) on the Na₂S-doped 8CB surface. After 15 min, 10 μL, was withdrawn from the aqueous droplet, which was then placed in 990 μL, of water and centrifuged at 10,000 rpm for 3 min using an accuSpin Micro 17R microcentrifuge. After centrifugation, the aliquot was collected, and the final concentration of heavy metal ions was determined using an inductively coupled plasma-optical emission spectrometry (ICP-OES; Agilent 5800 ICP-OES) at selected wavelengths for different heavy metal ion solutions (328 nm, 214 nm, 223 nm, 238 nm, and 220 nm for Ag⁺, Cd²⁺, Cu²⁺, Fe³⁺ and Pb²⁺, respectively). Then, the remaining concentration of heavy metal ions was calculated and the corresponding change in concentration was used to estimate the percentage of the heavy metal ion is removed by the water droplet.

Supplementary Materials

Interfacial Energy Criterion for Liquid Crystal-Infused Porous Surfaces

As described above, the porous substrate of a liquid crystal-infused porous surface should be preferentially wetted by the liquid crystal rather than water. Otherwise, the liquid crystal lubricant would be displaced by water droplets, rendering the surface unstable for the experiments. Both ΔE₁ and ΔE₂ should be greater than zero to ensure that water does not dewet the liquid crystal from the porous substrate (see equations (1) and (2) above).

The contact angles and interfacial tensions of water and 8CB were measured on porous polyRM257 substrates using a contact angle goniometer, with data summarized in Table S1.

TABLE S1
Interfacial tension and contact angle measurements
of water and liquid crystals at porous substrates.
n = 3 for the means and standard deviations.

| | |
|---|---|
| θ_{w} (°) | 84.5 ± 1.7 |
| θ_{LC} (°) | 0 |
| γ_{w} (mN/m) | 72.8 ± 1.6 |
| γ_{w-LC} (mN/m) | 19.5 ± 1.1 |
| γ_{LC} (mN/m) | 33.4 ± 1.8 |

Substituting the data in Table 51 into equations (1) and (2), it was calculated that ΔE₁=+7.0 mJ/m² and ΔE₂=+65.9 mJ/m², assuming that the roughness factor r=1 (the porous surfaces have r>1, and hence these are conservative values). These results indicate that an 8CB film will remain stable on porous polyRM257 substrates via surface tension-induced capillary forces and will not be dewetted from the substrate by water droplets.

Liquid Crystal Wrapping Layer at Air-Water Interface on Droplets

In the experiments herein, the presence of a wrapping layer of nematic 8CB on water droplets at the nematic 8CB surface was observed, indicated by the colored surface of the water droplet in FIG. 2. To support this observation, the spreading coefficient (S) of nematic 8CB at the air-water interface was calculated as (Anand S et al. ACS Nano 2012, 6, 10122-10129):

S=γ_{w}−(γ_{w-LC}+γ_{LC}) (S1)

A wrapping layer is formed when S >0 (Bonn D et al. Rev. Mod. Phys. 2009, 81, 739-805; Kreder M J et al. Phys. Rev. X 2018, 8, 031053). Using the data in Table 51 along with γ_{w}=72.8 mN/m, it was calculated that S=+19.9 mN/m, which confirms the presence of a nematic 8CB wrapping layer on water droplets at nematic 8CB surfaces. Next, the wrapping layer's equilibrium thickness (B) was estimated through equating the disjoining pressure (A_{H}/6πB³, where AH is the Hamaker constant for the air-liquid crystal-water interface) and the capillary pressure (2γ_{LC}/R). The disjoining pressure originates from van der Waals forces between molecules at the water-liquid crystal and liquid crystal-air interfaces (Li E Q et al. Langmuir 2014, 30, 5162-5169), while the capillary pressure arises due to the curvature of the droplet. B can be calculated as (Schellenberger F et al. Soft Matter 2015, 11, 7617-7626):

$$B = {( \frac{{A}_{H} R}{12 π {γ}_{L C}} )}^{1 / 3} \qquad ( S 2 )$$

Typically, A_{H}≈5×10⁻²⁰ J for hydrocarbons interacting with water or air (Israelachvili J N. Intermolecular and Surface Forces (Elsevier, 2011)). For a 10 μL water droplet (R˜1.8 mm), equation (S2) was used to calculate B˜41 nm, which is on the same order of magnitude as the thickness of the wrapping layer of isotropic lubricants on water droplets (Schellenberger F et al. Soft Matter 2015, 11, 7617-7626). The thickness of this wrapping layer cannot be deduced using standard optical techniques such as single wavelength reflection interference contrast microscopy (RICM) due to the non-constant refractive index of 8CB at the air-water interface (which originates from the degenerate planar anchoring of 8CB) (Concellon A et al. J. Am. Chem. Soc. 2019, 141, 18246-18255).

Impact of Mechanically Removed Liquid Crystal Lubricant on Gravimetric Measurements

In the gravimetric measurements, water droplets were removed from liquid crystal surfaces to measure the mass of liquid crystal surfaces after release of cargos. Specifically, droplets were removed by tilting a surface beyond the droplet's sliding angle, causing the droplet to slide off the surface and onto a glass slide. However, it is possible that some of the cargo-doped liquid crystal lubricant was collected with the water droplets, which would have caused overestimation of the mass of cargo released at the liquid crystal surface. To determine how significantly this could have impacted the results, the mass of the liquid crystal collected from the surface when removing a water droplet was measured. In these measurements, water droplets (10 μL) were collected from nematic 8CB surfaces in the same manner as before, dried on a glass slide, and the leftover liquid crystal was measured to be 0.016 mg/4 of water droplet. With this in mind, a typical thermal-triggered cargo release experiment was considered, where 80 μL of 8CB consisting of 20 wt % cargo microdroplets was coated on a 2.5 cm×2.5 cm polymerized polyRM257/8CB substrate. Then, a 10 μL water droplet was placed on top of the surface and the release of cargo from the liquid crystal surface into the droplet was induced. It was assumed that approximately 90% of the cargo was released, which corresponds to a mass of ˜14.4 mg. The mass loss of 8CB was just 0.16 mg for the 10 μL water droplet, which is two orders of magnitude smaller than the release of cargo. Therefore, it was concluded that the contribution of the lubricant removed with the droplet did not obscure the analyses. The mass of the liquid crystal wrapping layer with B˜41 nm (calculated previously using equation (S2)) is ˜4.6×10⁻⁵ mg/μL of water droplet, which is three orders of magnitude lower than the measured mass (˜0.016 mg/μL of water droplet) of mechanically removed lubricant. This result suggests that the mass loss of liquid crystal lubricant associated with removal of water droplets occurs mainly due to the removal of wetting ridges (rather than the wrapping layers) of the water droplet, which is consistent with previous reports of isotropic oil-based SLIPS systems (Kreder M J et al. Phys. Rev. X 2018, 8, 031053).

Liquid Crystal Mesophase-Dependent Static Friction Force

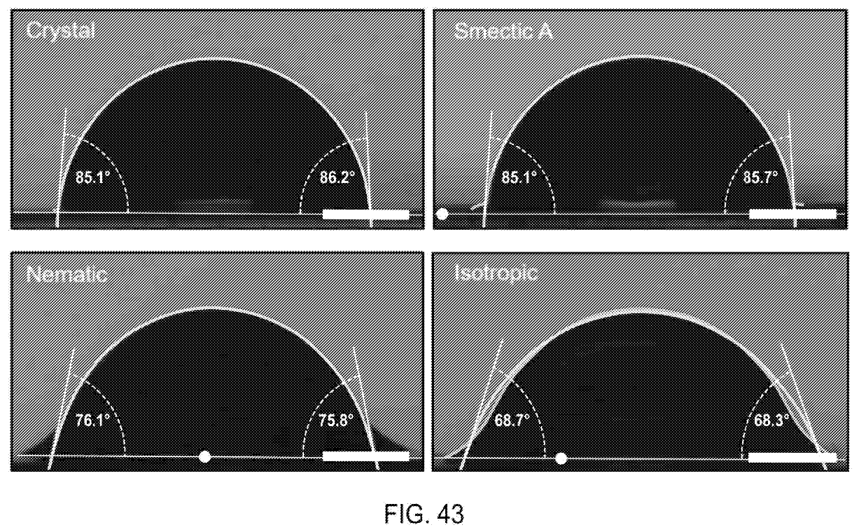

The apparent advancing contact angle (Oath) of water droplets at the 8CB surface for various liquid crystal phases was reported above. As shown in FIG. 3 and FIG. 43, θ_{adv} of static water droplets at the 8CB surface were similar over a wide temperature range (5-50° C.). The force of static friction (F_{static}) is governed by contact line pinning (Xu W et al. Phys. Rev. Lett. 2012, 109, 024504; Forsberg P S et al. Langmuir 2010, 26, 860-865), which is caused by contact angle hysteresis (the difference between apparent advancing and receding contact angles) (Semprebon C et al. Soft Matter 2016, 13, 101-110; Lafuma A et al. Europhys. Lett. 2011, 96, 56001). For water droplets at 8CB surface (Liu M et al. Nat. Rev. Mater. 2017, 2, 17036; de Gennes P G et al. Capillarity and Wetting Phenomena (Springer, 2004); Quere D. Annu. Rev. Mater. Sci. 2008, 38, 71-99):

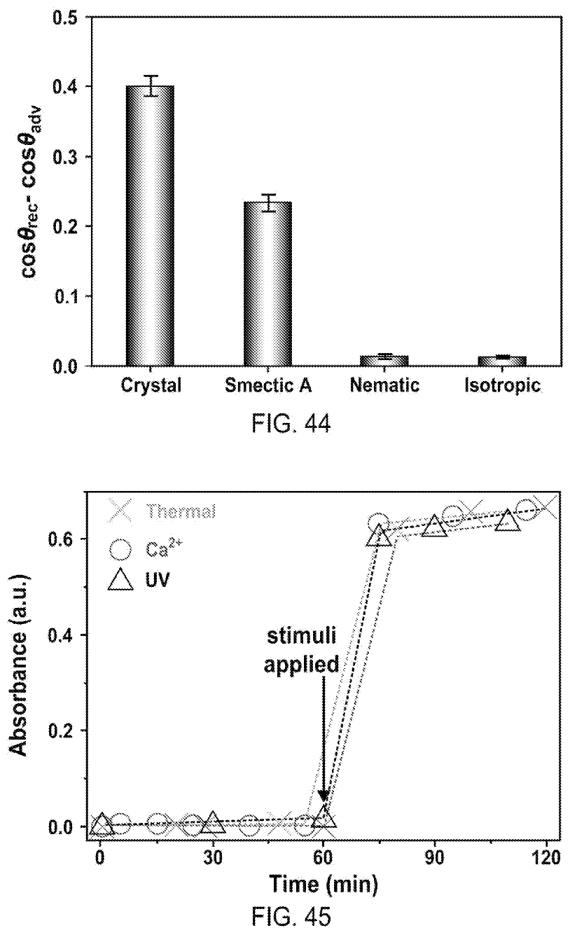

F_{static}=wγ_{w-LC}(cos θ_{rec}−cos θ_{adv}) (S3)

in which w is the width of the droplet and θ_{adv} and θ_{rec} are the apparent advancing and receding contact angles, respectively. Using the values of F_{static} from FIG. 3, equation (S3) (with γ_{w-LC} from Table S1) can be used to calculate contact angle hysteresis (defined as cos θ_{rec}−cos θ_{adv}) of water droplets at the 8CB surface. As shown in FIG. 44, the contact angle hysteresis of 3 μL droplets decreased from ˜0.40 in the crystalline phase to ˜0.23 in the sematic A phase, down to ˜0.02 in both the nematic and isotropic phases. Previous studies have demonstrated that surface roughness affects the contact angle hysteresis of liquid droplets, especially on rough surfaces (de Gennes P G et al. Capillarity and Wetting Phenomena (Springer, 2004)). It is reasoned here that as 8CB transitions from the crystalline phase (three dimensional positional order) to the smectic A phase (one dimensional positional order) and then further to the nematic and isotropic phases (both with no positional order), the decrease in mesogenic positional order leads to a decrease in the roughness of 8CB surfaces, which in turn decreases the contact angle hysteresis of water droplets. This result lead to the conclusion that positional order greatly influences the static force of friction that acts on water droplets on liquid crystal surfaces.

Liquid Crystal Mesophase-Dependent Dynamic Friction Force

In addition to F_{static}, F_{dynamic} of water droplets on 8CB surfaces was measured. As shown in FIG. 4, water droplets were highly pinned at the smectic A 8CB surface (F_{static}=16.2 μN) with F_{static}>F_{dynamic}. This is characteristic of the stick-slip mode (Gao N et al. Nat. Phys. 2017, 14, 191-196). In contrast, water droplets at the nematic liquid crystal surface exhibited a small F_{static} of 0.6 μN with F_{static} F_{dynamic}, which is consistent with the slippery mode (Smith J D et al. Soft Matter 2013, 9, 1772-1780; Daniel D et al. Nat. Phys. 2017, 13, 1020-1025). In addition, it was found that F_{dynamic} of water droplets increases as droplet velocity U increases from 0.1 to 1.0 mm/s, as shown in FIG. 5. Previous studies have demonstrated that F_{dynamic} of droplets moving across isotropic lubricant-based slippery lubricant-infused porous surfaces (SLIPS) with isotropic lubricants can be predicted by Landau-Levich-Derjaguin (LLD) theory for dip-coating (Daniel D et al. Nat. Phys. 2017, 13, 1020-1025). According to LLD theory, the thickness of the lubricant film underneath water droplets increases as the dimensionless capillary number (which is proportional to U) increases (Daniel D et al. Nat. Phys. 2017, 13, 1020-1025):

h_{film}˜R Ca^{2/3}=R(ηU/γ_{w-1})^{2/3} (S4)

in which h_{film} is the thickness of lubricant underneath water droplets, Ca is the capillary number (which compares viscous and capillary forces), η is the viscosity of the lubricant, and γ_{w-1} represents the water-lubricant interfacial tension (for liquid crystal surfaces, γ_{w-1}=γ_{w-LC}). F_{dynamic} can be calculated by integrating the viscous stress (ηU/h_{film}) over the droplet's rim area 2πRl, where l is the length of the contact line, as (Daniel D et al. Nat. Phys. 2017, 13, 1020-1025):

F_{dynamic}≈ηU/h_{film}2πRl≈2πγ_{w-1}R Ca^{2/3} (S5)

Equation (S5) is the same as equation (3) above. As shown in FIG. 6, the experimentally measured F_{dynamic} is linear with respect to the theoretically determined F_{dynamic} calculated using equation (S5), suggesting that droplets sliding on nematic liquid crystal surfaces follow LLD behavior for dip-coating and that there is no significant contact line pinning of water droplets on nematic liquid crystal surfaces. In addition, the straight line fits the data for a ˜20 cP viscosity, which is consistent with degenerate planar anchoring of nematic 8CB at aqueous interfaces (Chmielewski A G. Mol. Cryst. Liq. Cryst. 1986, 132, 339-352).

Optical Manipulation of Water Droplets at Azobenzene-Doped Liquid Crystal Surfaces

The results above indicate that the positional order of the liquid crystal greatly affects static friction of water droplets on liquid crystal surfaces. Next, the use of UV light to reversibly switch the positional order of the liquid crystal to manipulate the mobility of millimeter-size water droplets at the liquid crystal surface was investigated. In the experiments, 1 wt % azobenzene was mixed with 8CB, causing the smectic A to nematic (SmA-N) transition temperature to decrease by 2.0° C. to ˜31.7° C. (FIG. 7). Then, a Linkam PE120 Peltier hot plate was used to maintain the surface temperature of the 8CB porous substrate at 30.5° C. As shown in FIG. 8, the surface was inclined by 3°, as measured from the horizon. When a 3 μL water droplet was placed at the azobenzene-doped 8CB surface, the droplet remained pinned (stick-slip mode) when the surface was in the smectic phase. Upon exposure to UV light (365 nm wavelength), trans-cis isomerization of azobenzene induced a smectic A-nematic phase transition of 8CB, causing the droplet to enter the slippery mode and slide across the nematic 8CB surface. When the UV exposure was turned off and on, the water droplet repeatedly switched between the stick-slip and slippery modes, thereby causing the droplet to alternate between moving freely without noticeable pinning and being pinned at the 8CB surface.

Next, a photomask was used to selectively expose the liquid crystal surface to UV radiation to manipulate the positions of water droplets at the liquid crystal surface. The azobenzene-doped 8CB surface was inclined to 30° above the horizontal. As shown in FIG. 9, water droplets (3 μL) of two different colors (red and dark blue) were placed at opposite ends of the azobenzene-doped 8CB surface. Next, aluminum foil, which is opaque to UV light, was used to cover the bottom of the surface (red droplets) and the top of the surface (dark blue droplets) was exposed to UV radiation. After 3 min of UV exposure, the red water droplets remained immobile, whereas the dark blue water droplets at the UV-exposed liquid crystal surface (which transitioned to the nematic phase) freely slid to the edge of the region covered by the aluminum foil.

Moreover, the use of selective exposure of UV radiation to control the trajectory of water droplets sliding down an inclined 8CB surface at an inclination angle of 30° from the horizontal position was investigated. As shown in FIG. 10, after exposing the entirety of an azobenzene-doped 8CB surface to UV radiation, droplets deposited at the 8CB surface slid straight down due to gravity. Next, visible light was used to trigger the cis-trans isomerization of azobenzene, thereby transitioning 8CB from nematic to smectic A, resulting in pinning of water droplets at the 30° inclined 8CB surface. Next, the 8CB surface was selectively exposed to UV radiation, and it was observed that the trajectory of the water droplet sliding down the inclined surface followed the edge of the region covered by aluminum foil, demonstrating directional mobility. These results show that the UV radiation pattern could be reversibly written and erased to guide the path of water droplets sliding down an inclined 8CB surface, which offers a multitude of possibilities that enable interfacial transport of water droplets in a programmable manner.

Automatic Release of Nano-Sized Cargo on Liquid Crystal Surfaces

As discussed above, nano-sized ethyl orange (EO) aggregates were loaded in the bulk of the 8CB film by using ethanol as a co-solvent, which was subsequently evaporated before further experiments. UV-visible spectrophotometry was used to measure the amount of the ethyl orange nanocargo released into water droplets placed at the liquid crystal-infused porous surfaces, as shown in FIG. 11. It was observed that the nano-sized ethyl orange aggregates automatically released to water droplets at the 8CB surface, where they became saturated after around 8 min. This release profile is distinct from that of liquid crystal surfaces loaded using the inverse emulsion-based method (which followed release triggered by external stimuli). These results indicate that the energy barrier associated with release of nano-sized cargos at liquid crystal surfaces is negligible.

Size-Dependent Liquid Crystal Ordering Around Encapsulated Cargos in Liquid Crystal

As shown in FIG. 13-FIG. 15, cargo size-dependent release behavior was observed at liquid crystal surfaces. To qualitatively support these observations, polarized light microscopy imaging of liquid crystal-dispersed cargos prepared by two different loading methods (see “Methods of loading cargo into 8CB surfaces” above) was performed. As shown in FIG. 40, nano-sized ethyl orange cargos prepared by the co-solvent method caused no change in the optical appearance of unidirectionally-aligned nematic 8CB, whereas ethyl orange microdroplets prepared by the inverse emulsion method caused elastic distortion of the liquid crystals surrounding the droplets. Specifically, anionic surfactant sodium dodecyl sulfate (SDS) adsorbed at the aqueous-8CB interfaces caused perpendicular surface anchoring of nematic 8CB, resulting in a point topological defect surrounding each aqueous microdroplet (Kim Y K et al. Nature 2018, 557, 539-544; Guo J K et al. Adv. Sci. 2019, 6, 1900785). The above observations of loading method-dependent release profiles on liquid crystal surfaces can be attributed to the size-dependent liquid crystal ordering around the guest cargo (Yamamoto J et al. Nature 2001, 409, 321-325; Urbanski M et al. J. Phys. Condens. Matter 2017, 29, 133003), which is determined by a competition between the elastic energy (KR cargo, where K is the Frank elastic constant and R_{cargo} is the radius of the cargo microdroplet) and surface anchoring energy (WR_{cargo}², where W is the surface anchoring strength) (Kiernan M et al. Soft Matter Physics: An Introduction (Springer, 2003)). Specifically, the local mesogenic orientation around microcargos (R_{cargo}>K/W˜1 μm) prevents their automatic release from the liquid crystal surface, whereas nano-sized cargos with R_{cargo}<K/W that cause no orientational elastic distortion automatically release.

UV-Visible Spectrophotometry Measurements of Activated Cargo Release at Liquid Crystal Surface

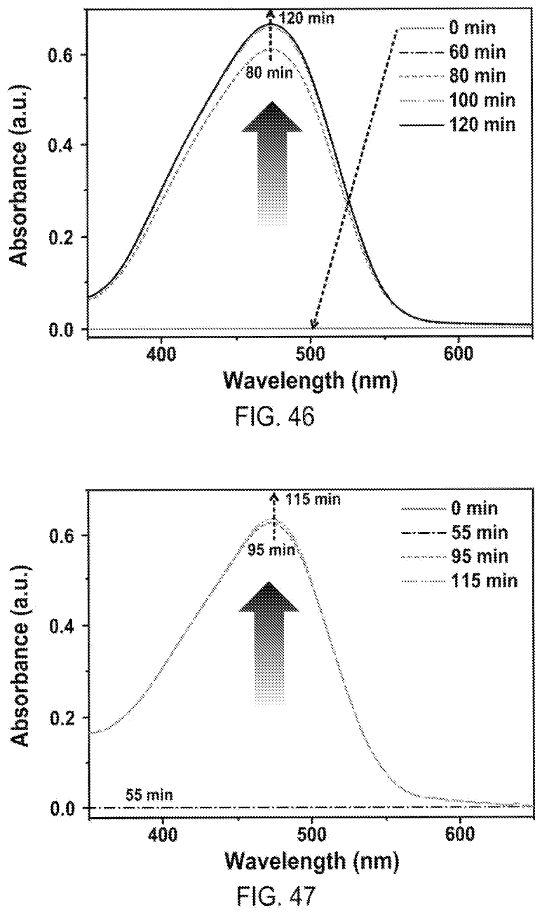

As described above, the release of ethyl orange aqueous microdroplets into water droplets at the 8CB surface can be triggered using a variety of stimuli. In addition to quantifying cargo release using gravimetry, UV-visible spectrophotometry was also used to measure the amount of ethyl orange dye released into the water droplets placed at the ethyl orange-doped nematic 8CB surface. Upon the nematic to isotropic (N-I) transition, it was observed that the ethyl orange microdroplets encapsulated in the 8CB surface released into the water droplet, as shown in FIG. 45 and FIG. 46. In addition to heat, it was demonstrated that charge can trigger the release of ethyl orange aqueous microdroplets at 8CB surface. As shown in FIG. 45 and FIG. 47, the attractive electric double layer interaction between the anionic surfactant SDS-adsorbed ethyl orange microdroplets and cationic aqueous Ca²⁺ overcame the elastic distortion of the nematic 8CB and activated the release of ethyl orange microdroplets.

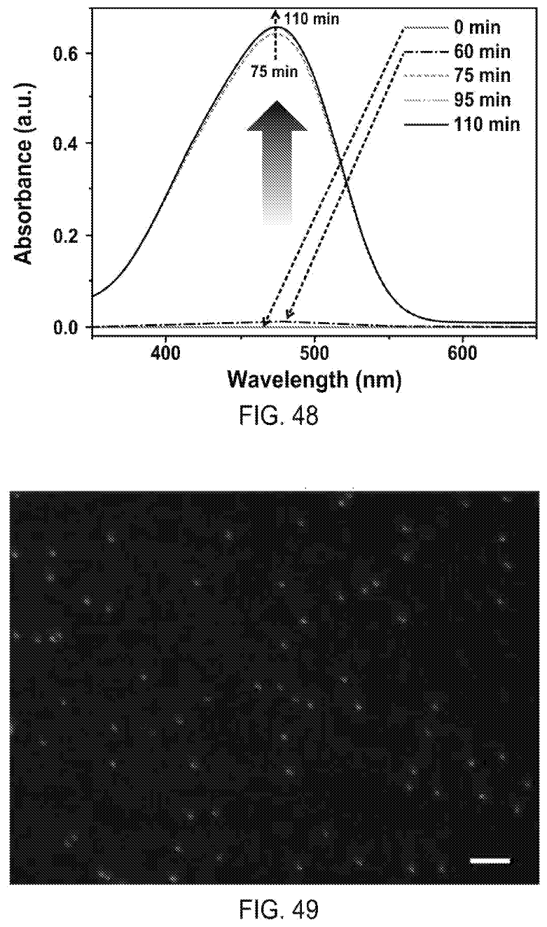

Beyond heat and charge, it was also demonstrated that UV light can activate the release of ethyl orange microdroplets at azobenzene-doped 8CB surfaces. In these experiments, 1 wt % of azobenzene was doped in 8CB, causing the nematic-isotropic phase transition temperature to decrease by 1° C. upon exposure to UV radiation (T_{N-1}=˜38.5° C. without UV and ˜37.5° C. with UV). A Linkam PE120 Peltier hot plate was used to maintain the surface temperature of the 8CB-infused porous substrate at 38° C. As shown in FIG. 45 and FIG. 48, upon UV radiation, the 8CB surface underwent a nematic-isotropic phase transition, which activated the release of ethyl orange microdroplets to water droplets at the liquid crystal surface. These observations demonstrate that orientational order of liquid crystals can be manipulated to enable activated cargo release from liquid crystal surfaces to water droplets.

Thermodynamic Model for Activated Cargo Release at Liquid Crystal Surface

The experimental observations in FIG. 13 and FIG. 14 suggest that either a nematic-isotropic transition (induced by heat or UV) or the presence of Ca²⁺ can activate the release of cargo microdroplets (an aqueous solution of SDS and ethyl orange) to millimeter-sized water droplets at 8CB surfaces. Here, the derivation of a simple thermodynamic model to describe the key features of this activated release behavior was sought. In Sections 1-5, each interaction involved in the process of activated release of cargo microdroplets, including the van der Waals force, capillary force, electric double layer force, liquid crystal elastic force, and buoyancy force, is described. In Section 6, these forces are combined into a thermodynamic model to describe the essential behaviors of droplets interacting with liquid crystal surfaces during the process of activated cargo release.

Section 1. van der Waals force: The attractive van der Waals forces (F_{vdW}) between the cargo microdroplet and the millimeter-sized water droplet can be written as (Israelachvili J N. Intermolecular and Surface Forces (Elsevier, 2011)):

$${F}_{v d W} = - \frac{{A}_{H} R {R}_{c a r g o}}{6 {x}^{2} ( {R}_{c a r g o} + R )} \qquad ( S 6 )$$

where R_{cargo} is the radius of the cargo microdroplet and x is the surface-to-surface distance between the cargo microdroplet and millimeter-sized water droplet. In these calculations, it was assumed that R_{cargo}=5 μm and R=1.75 mm. For R_{cargo}<<R, equation (S6) can be simplified to:

$${F}_{v d W} = - \frac{{A}_{H} {R}_{c a r g o}}{6 {x}^{2}} \qquad ( S7 )$$

The negative sign in equation (S7) indicates that F_{vdW} is attractive in the process of cargo release (A_{H} is always positive).

Section 2. Capillary force: As described above, the capillary force induced by the curved capillary meniscus (e.g., wetting ridge) surrounding the water droplets provides a driving force which concentrates the cargo microdroplets in the wetting ridge of the millimeter-sized water droplet. To gain insight into the role of the capillary force in the process of activated cargo release, derivation of an expression to estimate the capillary force was sought.

At any arbitrary point in a curved liquid crystal surface, the local hydrostatic pressure P can be deduced from the Laplace pressure (de Gennes P G et al. Capillarity and Wetting Phenomena (Springer, 2004)):

$$P = {P}_{atm} - {γ}_{L C} ( \frac{1}{{R}_{1}} - \frac{1}{{R}_{2}} ) \qquad ( S8 )$$

where Pₐₜₘ is the pressure of atmosphere, and R₁ and R₂ are the two principal radii of curvature (as shown in FIG. 17).

From Equation (S8), it can be written that the hydrostatic pressure in the liquid crystal wetting ridge (P_{ridge}) as:

$${P}_{ridge} = {P}_{a t m} - {γ}_{L C} ( \frac{1}{{R}_{1}} - \frac{1}{R} ) \qquad ( S9 )$$

For SLIPS systems, R>R₁, which suggests Pₐₜₘ>P_{ridge}. This analysis indicates that the capillary force induced by hydrostatic pressure gradient associated with liquid crystal wetting ridge provides a driving force to concentrate cargo microdroplets within the liquid crystal wetting ridge around the millimeter-sized water droplet at the liquid crystal surface. The distance that capillary force induced by the wetting ridge dominates over gravitational force can be estimated as the capillary length (de Gennes P G et al. Capillarity and Wetting Phenomena (Springer, 2004)):

capillary length=\sqrt{γ_{LC}/(ρ_{LC}g)} (S10)

in which ρ_{LC} is the mass density of the liquid crystal (1.02 g/cm 3) (Kim J W et al. Langmuir 2004, 20, 8110-8113) and g is the acceleration of gravity (9.8 m/s 2). From equation (S10), the capillary length is estimated to be ˜1.8 mm.

For cargo microdroplets encapsulated inside the curved liquid crystal surface, the average hydrostatic pressure experienced on the hemispheres of cargo microdroplets toward and away from the millimeter-sized water droplet can be written as:

$${P}_{c a r g o , 1} = {P}_{atm} - {γ}_{L C} ( \frac{1}{{R}_{1}} - \frac{1}{R + x - ξ {R}_{c a r g o}} ) \qquad ( S11 )$$

$${P}_{c a r g o 2} = {P}_{atm} - {γ}_{L C} ( \frac{1}{{R}_{1}} - \frac{1}{R + x + ξ {R}_{c a r g o}} ) \qquad ( S12 )$$

where ξ is a coefficient to estimate the average hydrostatic pressure acting on each hemisphere (ξ=2/3).

From equations (S11) and (S12), the hydrostatic pressure gradient (ΔP) around the cargo microdroplet can be written as:

$$Δ P = {P}_{c a rgo , 2} - {P}_{c a r g o , 1} \approx {γ}_{L C} ( \frac{1}{R + x + ξ {R}_{c a r g o}} - \frac{1}{R + x - ξ {R}_{c a r g o}} ) = \frac{- 2 {γ}_{L C} ξ {R}_{c a r g o}}{{( R + x )}^{2} - {( ξ {R}_{cargo} )}^{2}} \approx \frac{- 2 {γ}_{L C} ξ {R}_{c a r g o}}{{( R + x )}^{2}} ( for R ≫ {R}_{c a r g o} ) \qquad ( S13 )$$

By multiplying AP by the cross-sectional area of cargo microdroplet, the capillary force (F_{cap}) is obtained:

$${F}_{c a p} = Δ P π {R}_{c a r g o}^{2} = - \frac{2 {γ}_{L C} π ξ {R}_{c a r g o}^{3}}{{( R + x )}^{2}} \qquad ( S14 )$$

The negative sign in equation (S14) indicates that F_{cap} is always attractive and acts as a driving force in the process of cargo release.

Section 3. Electric double layer force: For R_{cargo}<<R, the electric double layer force (F_{edl}) between the cargo microdroplet and the millimeter-sized water droplet can be written as (Israelachvili J N. Intermolecular and Surface Forces (Elsevier, 2011)):

F_{edl}=64πε₀ε_{LC}(k_{B}T/e)² tan h[zeψ_{cargo}/(4k_{B}T)] tan h[zeψ_{drop}/(4k_{B}T)]κR_{cargo}e^{-κx} (S15)

where ε₀ is the vacuum permittivity (ε₀=8.854×10⁻¹² C/V m), ε_{LC} is the relative permittivity of the liquid crystal (ε_{LC}=10) (Kreul H et al. Phys. Rev. A: At., Mol., Opt. Phys. 1992, 45, 8624-8631), k_{B} is the Boltzmann constant (k_{B}=1.38×10⁻²³ J/K), T is the temperature, z is the valence number of the dominant aqueous ionic species, e is the elementary charge (e=1.6×10⁻¹⁹ C), κ⁻¹ is the Debye length, and ψ_{cargo} and ψ_{drop} are the zeta potentials of cargo microdroplet and millimeter-sized water droplet, respectively. K 1 was assumed to 1.2 μm in liquid crystal without added electrolytes (Shah R R et al. J. Phys. Chem. B 2001, 105, 4936-4950). 1i/cargo of 5 mM SDS aqueous droplets of ethyl orange was set to −100 mV (Kim Y K et al. Nature 2018, 557, 539-544), whereas ψ_{drop} of millimeter-sized water droplets of pure water and Ca²⁺ solution was set to −20 mV (Manilo M V et al. J. Mol. Liq. 2019, 276, 875-884) and +100 mV (Kim Y K et al. Nature 2018, 557, 539-544), respectively. T was set to 308 K and 318 K for the nematic and isotropic phases, respectively. z was set to 1 for millimeter-sized water droplet and SDS cargo microdroplets and 2 for millimeter-sized droplet of Ca²⁺. F_{edl} can be either attractive or repulsive: F_{edl} is repulsive if ψ_{cargo} and ψ_{drop} have the same sign, whereas F_{edl} is attractive if they have different signs.

Section 4. Liquid crystal Elastic force: In bulk liquid crystal, a repulsive interaction caused by long-range orientational ordering of the liquid crystal arises when the dispersed colloidal particles move towards a surface (Poulin P et al. Science 1997, 275, 1770-1773; Pishnyak O P et al. Phys. Rev. Lett. 2007, 99, 127802; Chernyshuk S B et al. Phys. Rev. E 2011, 84, 011707). The liquid crystal elastic force (Fₑₗ) between the cargo microdroplet and millimeter-sized water droplet interface can be calculated as (Chernyshuk S B et al. Phys. Rev. E 2011, 84, 011707):

$${F}_{el} = + \frac{{α}^{2} β π K {R}_{c a r g o}^{4}}{{( {R}_{c a r g o} + x )}^{4}} \qquad ( S16 )$$

where α and β denote constants related to liquid crystal, and K denotes the Frank elastic constant of liquid crystal. α is 2.04 for the cargo droplet with homeotropic anchoring in nematic phase when R_{cargo}>K/W, and 0 for cargo either in isotropic phase or R_{cargo}<K/W in nematic phase (Chernyshuk S B et al. Phys. Rev. E 2011, 84, 011707). β=1/2 for homeotropic anchoring (Kim Y K et al. Nature 2018, 557, 539-544). K is set to 5 pN (Kléman M et al. Soft Matter Physics: An Introduction (Springer, 2003)). The maximum Fₑₗ is 32.7 pN, calculated by equation (S16) with x=0. The positive sign in equation (S16) indicates that the liquid crystal elastic force is always repulsive and prevents the release of cargo microdroplets.

Section 5. Buoyancy force: The magnitude of the buoyancy force (Fb) acting on the cargo microdroplet can be written as:

F_{b}=(4/3)πR_{cargo}³g(ρ_{LC}−Σ_{water}) (S17)

in which ρ_{water} is the density of water (ρ_{water}=1.0 g/cm³). From equation (S17), F_{b} acting on microdroplet is ˜0.1 pN, which is two orders of magnitude smaller than the maximum value of Fₑₗ, which leads to the conclusion that the contribution of buoyancy force on the cargo microdroplet can be neglected.

Section 6. Net force: In this section, the above forces are combined into a single model to describe the key features of the activated release behavior at liquid crystal surfaces. From equations (S7), (S14), (S15), and (S16), the net force (Fₙₑₜ) acting on the cargo microdroplet can be written as (same as equation (4)):

$${F}_{net} = {F}_{vdW} + {F}_{cap} + {F}_{edl} + {F}_{el} = - \frac{{A}_{H} {R}_{cargo}}{6 {x}^{2}} - \frac{2 {γ}_{LC} πξ {R}_{cargo}^{3}}{{( R + x )}^{2}} + 64 π {ε}_{0} {{ε}_{LC} ( {k}_{B} T / e )}^{2} tanh ( ze {ψ}_{cargo} / 4 {k}_{B} T ) tanh ( ze {ψ}_{drop} / 4 {k}_{B} T ) κ {R}_{cargo} {e}^{- κ x} + \frac{{α}^{2} β π {KR}_{cargo}^{4}}{{( {R}_{cargo} + x )}^{4}} \qquad ( S18 )$$

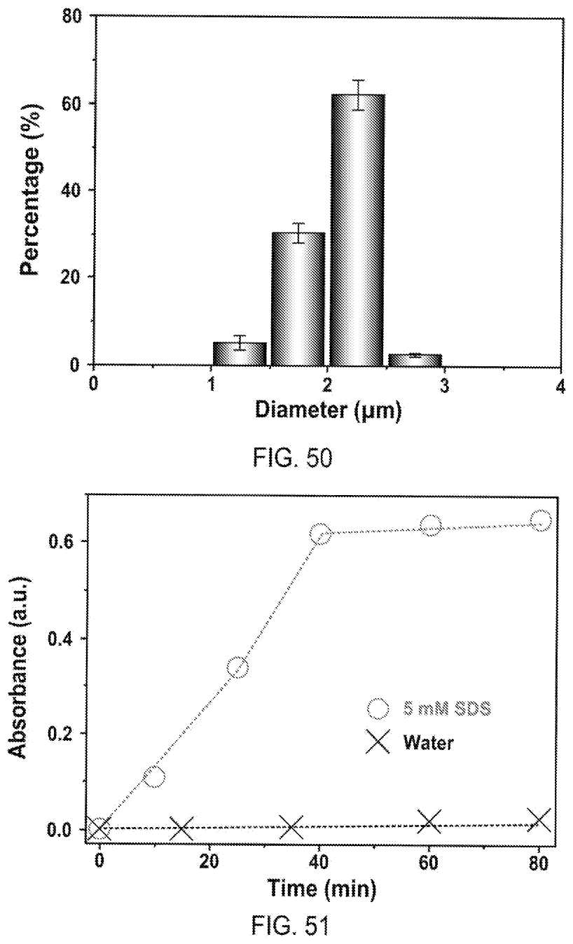

Formation of glycerol droplets during isotropic-nematic transition of 8CB. As described above, reversible switching of liquid crystal orientational order was taken advantage of to induce condensation of glycerol in bulk liquid crystal for reversible loading and release of cargos. Past studies have reported that glycerol molecularly diffuses in isotropic liquid crystals (Nazarenko V G et al. Phys. Rev. Lett. 2001, 87, 075504). However, increasing the mesogenic orientational order through an isotropic-nematic transition lowers the solubility of glycerol in the liquid crystal, resulting in the condensation and growth of glycerol microdroplets within the bulk of the liquid crystal. Herein, fluorescence microscopy imaging was performed to measure the size distribution of glycerol droplets formed during the isotropic-nematic phase transition. As shown in FIG. 49 and FIG. 50, the average diameter of glycerol droplets was 2.2 μm. Based on the observations of cargo size-dependent release behavior described above, it was concluded that the local mesogenic orientational order around microdroplets prevents their automatic release from the liquid crystal surface.

Automatic release of Rhodamine B-doped glycerol at isotropic 8CB surface. Based on observations of automatic release of nano-sized ethyl orange aggregates to water droplets at nematic 8CB surfaces (FIG. 17-FIG. 21), it was hypothesized that glycerol molecularly dispersed in isotropic 8CB surfaces can automatically release to water droplets. To validate this hypothesis, after loading the isotropic 8CB surface with glycerol (containing 20 mM Rhodamine B), the temperature of the 8CB surface was maintained at 45° C. (isotropic phase) and a water droplet was placed on the surface. As shown in FIG. 23 and FIG. 24, UV-visible spectrophotometry measurements show that Rhodamine B-doped glycerol automatically released into water droplets placed on the liquid crystal-infused porous surfaces, which was consistent with the hypothesized cargo size-dependent release behavior.

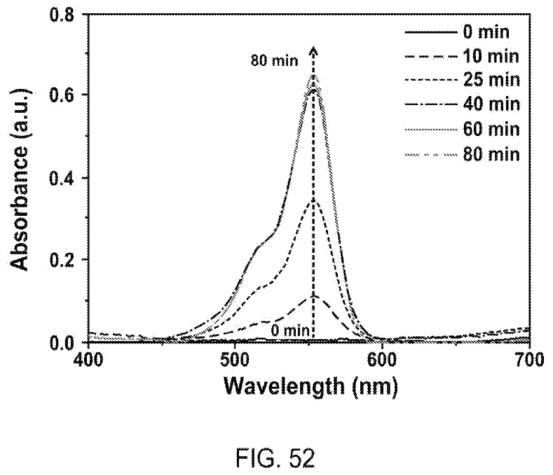

In-situ loading and release of cargo on liquid crystal surfaces. It was demonstrated above that reversible loading and release of glycerol could be achieved through reversible switching of the orientational order of liquid crystals. UV-visible spectrophotometry was also used to measure the amount of Rhodamine B released into water droplets deposited on nematic 8CB surfaces. As shown in FIG. 51 and FIG. 52, 5 mM SDS aqueous droplets can activate the release of Rhodamine B-doped glycerol droplets dispersed in nematic 8CB films.

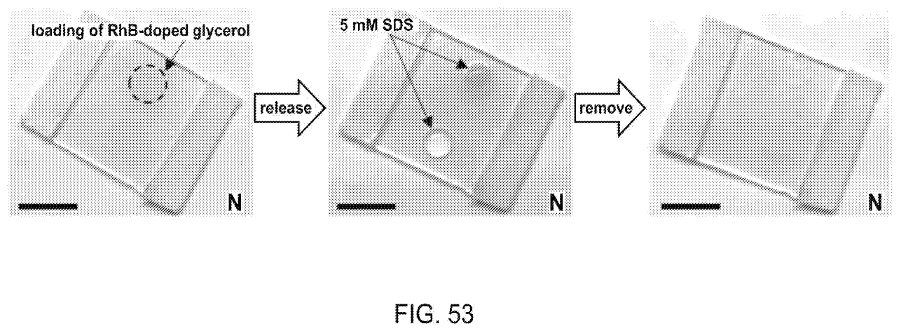

Next, demonstration that this method can also be used in-situ was sought. As shown in FIG. 53, Rhodamine B-doped glycerol was selectively loaded onto specific areas of the 8CB surface. Subsequently, SDS aqueous droplets were placed at both the cargo-loaded region and another region −0.75 cm away from the cargo-loaded region. As time progressed, it was observed that Rhodamine B-doped glycerol released into water droplets only at the areas where cargo had been loaded into the 8CB surface, without interference from other regions. These results imply that in-situ loading and release of cargo molecules can be achieved through selective loading and release from specific areas of the liquid crystal surface.

Photocatalytic degradation of various organics by TiO₂-loaded liquid crystal surfaces. It was demonstrated above that Rhodamine B in water can be degraded by TiO₂-mediated photocatalysis (FIG. 27-FIG. 31). Herein, whether this methodology is generalizable to other water-soluble organic dyes (e.g., ethyl orange, methyl orange, and methylene blue) was tested. In the experiments, TiO₂ nanoparticles dispersed in aqueous Brij 97 (0.8 mM) were mixed with 8CB at 35° C., then 80 μL of the obtained inverse emulsion was coated onto a 2.5 cm×2.5 cm porous polyRM257 substrate. Next, 10 μL aqueous droplets with 0.6 mM of each dye were deposited on the 8CB surface and the release of TiO₂ was activated by inducing the nematic-isotropic phase transition of 8CB by increasing the temperature to 45° C. After cooling the surface down to 35° C. (nematic phase), aqueous droplets (which were initially colored due to the presence of the dye) turned colorless upon UV radiation for 3 min as the dye was degraded by TiO₂-mediated photocatalytical chemistry (Liu K et al. Chem. Rev. 2014, 114, 10044-10094), as shown in FIG. 30. This demonstrates that photocatalysis using TiO₂-loaded liquid crystal surfaces is a new generalizable technique for degrading organic compounds in water and other fluids.

Metal ion concentration-dependent F_{d} on liquid crystal surfaces. It was demonstrated above that S²⁻-loaded 8CB surfaces was able to self-sense and self-clean heavy metal ions, such as Pb²⁺ and Cd²⁺ from water droplets. Herein, the effect of metal ion concentration on F_{static} of aqueous droplets on liquid crystal surfaces was studied. As shown in FIG. 32, F_{static} increased by a factor of ˜10 when Pb²⁺ concentration increased from 0 to 50 mM. This can be attributed to an increase of contact line pinning of Pb²⁺ droplets at the nematic 8CB surface. Next, contact angle hysteresis of water droplets at nematic 8CB surfaces was calculated through substitution of F_{static} (FIG. 32) and interfacial tension data (Table S2) into equation (S3).

TABLE S2
Wettability of Pb²⁺ aqueous droplets at nematic 8CB surface.
n = 3 for means and standard deviations.

| Pb²⁺ concentration (mM) | θ_{adv} (°) | R (mm) | γ_{w-LC} (mN/m) |
|---|---|---|---|
| 0 | 84.5 ± 1.7 | 0.89 ± 0.10 | 19.5 ± 1.1 |
| 10 | 77.9 ± 1.8 | 0.90 ± 0.15 | 21.1 ± 0.9 |
| 20 | 69.7 ± 1.0 | 0.93 ± 0.20 | 25.0 ± 1.1 |
| 30 | 66.1 ± 0.9 | 0.98 ± 0.08 | 26.7 ± 1.2 |
| 40 | 65.1 ± 1.2 | 1.05 ± 0.13 | 29.2 ± 1.1 |
| 50 | 55.5 ± 1.5 | 1.10 ± 0.11 | 31.0 ± 1.2 |

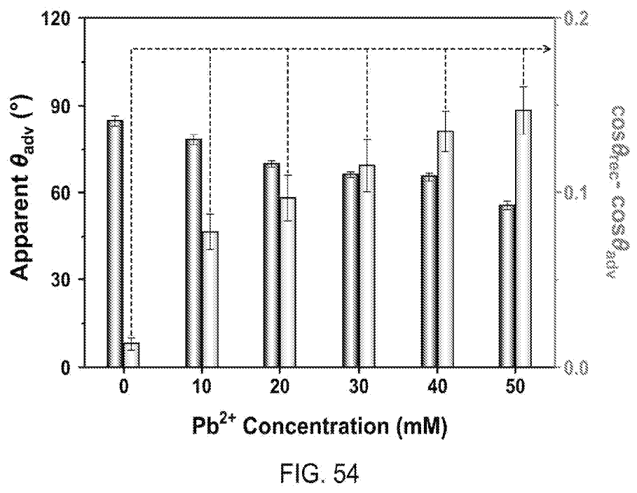

As shown in FIG. 54, the contact angle hysteresis of 3 μL droplets increased from 0.02 to 0.15 when increasing the concentration of Pb²⁺ from 0 to 50 mM, suggesting that the increase in F_{static} indeed stems from an increase in contact line pinning at the three-phase contact line when increasing the concentration of heavy metal ions in aqueous droplets. Next, the effect of metal ion concentration on F_{dynamic} of aqueous droplets at liquid crystal surfaces was investigate. As shown in FIG. 32, it was observed that F_{dynamic} increases as the concentration of Pb²⁺ increases. These results indicate that measuring F_{static} and F_{dynamic} at liquid crystal surfaces provides a new way to quantify the concentration of heavy metal ions in aqueous droplets.

It was hypothesized that this trend is caused by an increase in the viscous dissipative forces acting on droplets. The question of whether the impact of metal ions on F_{dynamic} behaves consistently with the LLD theory for dip-coating was explored. The data shown in FIG. 54 was substituted into equation (S5), which yielded a linear relationship between the calculated F_{dynamic} by LLD theory and the experimentally measured F_{dynamic} (FIG. 33), suggesting that the behavior of aqueous droplets containing metallic ions on 8CB surfaces are governed by LLD theory. In addition, the straight line best fit the data for ˜20 cP viscosity, indicating degenerate planar anchoring of nematic 8CB at the water droplet surface (Chmielewski A G. Mol. Cryst. Liq. Cryst. 1986, 132, 339-352).

### Example 2

The inherent molecular order of thermotropic liquid crystals has expanded their utility beyond display technologies and into the realm of functional surfaces that can sense chemical stimuli and template polymerization. However, water-induced instability of liquid crystal films has, until now, precluded the study of how molecular order affects an liquid crystal surface's slipperiness to millimeter-sized water droplets.

To address this long-standing challenge, porous liquid crystal polymeric networks are used herein to stabilize thermotropic liquid crystal mesogens against dewetting by water droplets, allowing the effect of mesogenic molecular order on the slipperiness of liquid crystal surfaces to be investigated. The stimuli-responsive characteristics of the liquid crystal-infused porous surfaces developed herein are based on the concept that: any environmental cue that can alter the molecular order of liquid crystals can cause the liquid crystal surface to respond. It was observed that external stimuli, including heat and light, are able to change the positional order of the mesogens, thus tuning the mobility of water droplets at the liquid crystal surface. Importantly, reversible, in-situ loading and release of cargos can be achieved via reversible switching of the orientational order of the mesogens in the liquid crystal surface.

Overall, the results reveal that both positional and orientational order of anisotropic, structured fluids can be selectively manipulated to influence both droplet mobility and cargo loading/release, thus providing a route for design of open surface microfluidics and smart surface reactors that enable self-sensing and self-cleaning of heavy metal ions in water.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A liquid crystal-infused porous surface, comprising:
a porous polymer layer having a surface, wherein the porous polymer layer comprises a continuous phase permeated by a plurality of pores, and wherein the continuous phase comprises a liquid crystal polymer; and
an anisotropic lubricant infused within and over the porous polymer layer, such that that the anisotropic lubricant at least partially fills the plurality of pores and forms a film on the surface of the porous polymer layer, wherein the anisotropic lubricant comprises thermotropic liquid crystal mesogen;
wherein the porous polymer layer without the anisotropic lubricant has a first total interfacial energy when wetted with water;
wherein:
when an aqueous droplet is disposed on the film of the anisotropic lubricant, then the liquid crystal-infused porous surface has a second total interfacial energy, and
in the absence of the aqueous droplet, then the liquid crystal-infused porous surface has a third total interfacial energy;
wherein the first total interfacial energy is greater than the second total interfacial energy; and
wherein the first total interfacial energy is greater than the third total interfacial energy.

2. The liquid crystal-infused porous surface of claim 1, wherein the liquid crystal polymer is derived from 1,4-Bis-[4-(3-acryloyloxypropyloxy)benzoyloxy]-2-methylbenzene (RM257), 4-(6-acryloxy-hex-1-yl-oxy) phenyl 4-(hexyloxy) benzoate, 4-methoxybenzoic acid 4-(6-acryloyloxyhexyloxy) phenyl ester 4″-acryloyloxybutyl 2,5-di(4′-butyloxybenzoyloxy) benzoate, or combinations thereof.

3. (canceled)

4. (canceled)

5. (canceled)

6. The liquid crystal-infused porous surface of claim 1, wherein the thermotropic liquid crystal mesogen comprises 4-cyano-4′-n-pentyl-biphenyl (5CB), 4-cyano-4′-n-heptyl-biphenyl (7CB), 4′-octyl-4-biphenylcarbonitrile (8CB), 4-cyano-4′-oxyoctyl-biphenyl (8OCB), 4-cyano-4′-n-pentyl-terphenyl (5CT), (S)-4-Cyano-4′-(2-methylbutyl)biphenyl (CB15), or a combination thereof.

7. (canceled)

8. (canceled)

9. The liquid crystal-infused porous surface of claim 1, wherein the liquid crystal-infused porous surface is disposed on a substrate.

10. (canceled)

11. The liquid crystal-infused porous surface of claim 1, wherein the thermotropic liquid crystal mesogen has:
a crystal mesophase when the thermotropic liquid crystal mesogen is at a temperature that is less than a first transition temperature, wherein when the thermotropic liquid crystal mesogen is in the crystal mesophase the thermotropic liquid crystal mesogen has long range orientational order and three dimensional positional order;
a smectic mesophase when the thermotropic liquid crystal mesogen is at a temperature greater than the first transition temperature and less than a second transition temperature, wherein the second transition temperature is greater than the first transition temperature, and wherein the smectic mesophase has long range orientational order and at least unidirectional positional order;
a nematic mesophase when the thermotropic liquid crystal mesogen is at a temperature greater than the second transition temperature and less than a third transition temperature, wherein the third transition temperature is greater than the second transition temperature, and wherein the nematic mesophase has long range orientational order and no positional order; and
an isotropic mesophase when the thermotropic liquid crystal mesogen is at a temperature above a second transition temperature, and wherein the isotropic mesophase has no orientational order and no positional order.

12. The liquid crystal-infused porous surface of claim 11, wherein an aqueous droplet having a volume placed on the film of the anisotropic lubricant is:
pinned when the thermotropic liquid crystal mesogen is in the crystal mesophase or the smectic mesophase; and
mobile when the thermotropic liquid crystal mesogen is in the nematic mesophase or the isotropic mesophase;
such that:
when the thermotropic liquid crystal mesogen is in the crystal mesophase or the smectic mesophase, then liquid crystal-infused porous surface is in a stick-slip mode; and
when the thermotropic liquid crystal mesogen is in the nematic mesophase or the isotropic mesophase, the liquid crystal-infused porous surface is in a slippery mode.

13. The liquid crystal-infused porous surface of claim 12, wherein the mobility of the aqueous droplet on the film of the anisotropic lubricant is temperature sensitive, as:
when the temperature of the thermotropic liquid crystal mesogen is increased from a temperature below the second transition temperature to a temperature above the second transition temperature, then the liquid crystal-infused porous surface transitions from the stick-slip mode to the slippery mode, such that the aqueous droplet becomes mobile; and
when the temperature of the thermotropic liquid crystal mesogen is decreased from a temperature above the second transition temperature to a temperature below the second transition temperature, then the liquid crystal-infused porous surface transitions from the slippery mode to the stick-slip mode, such that the aqueous droplet becomes pinned.

14. The liquid crystal-infused porous surface of claim 1, wherein the anisotropic lubricant further comprises a compound that can undergo photoisomerization.

15. (canceled)

16. (canceled)

17. The liquid crystal-infused porous surface of claim 14, wherein:
when the photoisomerization compound is a first photoisomer, the second transition temperature has a first value;
when the photoisomerization compound is a second photoisomer, the second transition temperature has a second value;
the first value is greater than the second value;
in the absence of a certain wavelength of light, the photoisomerization compound is the first photoisomer; and
when the photoisomerization compound is exposed to the certain wavelength of light, then the photoisomerization compound isomerizes to the second photoisomer; and
when the anisotropic lubricant is at a temperature above the first value and below the second value, then the mobility of the aqueous droplet on the film of the anisotropic lubricant is light sensitive as:
in the absence of the certain wavelength of light, the photoisomerization compound is the first photoisomer such that the second transition temperature has the first value, the thermotropic liquid crystal mesogen is in the smectic mesophase, the liquid-crystal infused porous surface is in the stick-slip mode, and the aqueous droplet is pinned; and
when the photoisomerization compound is exposed to the certain wavelength of light, then the photoisomerization compound isomerizes to the second photoisomer such that the second transition temperature has the second value, the thermotropic liquid crystal mesogen is in the nematic mesophase, the liquid crystal-infused porous surface is in the slippery mode, and the aqueous droplet is mobile.

18. (canceled)

19. The liquid crystal-infused porous surface of claim 17, wherein the liquid crystal-infused porous surface has a first portion and a second portion, wherein the first portion is selectively exposed to a certain wavelength of light and the second portion is not exposed to the certain wavelength of light, such that, when present, an aqueous droplet disposed on the first portion is mobile while an aqueous droplet disposed on the second portion is pinned.

20. The liquid crystal-infused porous surface of claim 19, wherein the first portion abuts the second portion along a border, and the border defines a path for motion of an aqueous droplet, when present.

21. The liquid crystal-infused porous surface of claim 1, wherein the anisotropic lubricant further comprises a cargo; wherein the anisotropic lubricant further comprises a plurality of droplets comprising a cargo; or a combination thereof.

22. (canceled)

23. The liquid crystal-infused porous surface of claim 21, wherein the cargo comprises an organic species, a photocatalyst, a heavy metal ion capture species, a medicament, a drug, or a combination thereof.

24-30. (canceled)

31. The liquid crystal-infused porous surface of claim 21, wherein the anisotropic lubricant further comprises a plurality of droplets comprising a cargo and wherein:
the plurality of droplets comprising the cargo have an average radius, an average elastic energy, and an average a surface anchoring energy;
the elastic energy is equal to the product of the average radius of the plurality of droplets and the Frank elastic constant;
the average surface anchoring energy is equal to the product of the average radius of the plurality of droplets squared and the surface anchoring strength;
when the thermotropic liquid crystal mesogen is in the isotropic mesophase and an aqueous droplet is placed on the film of anisotropic lubricant, at least a portion of the plurality of droplets comprising the cargo are automatically released from the anisotropic lubricant into the aqueous droplet;
when an aqueous droplet is placed on the film of anisotropic lubricant and the thermotropic liquid crystal mesogen is in the nematic mesophase, at least a portion of the plurality of droplets comprising the cargo are automatically released from the anisotropic lubricant into the aqueous droplet when the surface anchoring energy is less than or equal to the elastic energy; and
when the aqueous droplet is placed on the film of anisotropic lubricant and the thermotropic liquid crystal mesogen is the in the nematic mesophase, substantially none of the plurality of droplets comprising the cargo are released from the anisotropic lubricant into the aqueous droplet when the surface anchoring energy is greater than the elastic energy.

32. The liquid crystal-infused porous surface of claim 31, wherein:
the surface anchoring energy is less than or equal to the elastic energy when the average radius of the plurality of droplets comprising the cargo is less than or equal to the quotient of the Frank elastic constant and the surface anchoring strength;
the surface anchoring energy is greater than the elastic energy when the average radius of the plurality of droplets comprising the cargo is greater than the quotient of the Frank elastic constant and the surface anchoring strength;
the surface anchoring energy is less than or equal to the elastic energy when the average radius of the plurality of droplets comprising the cargo is 1 micrometer or less;
the surface anchoring energy is greater than the elastic energy when the average radius of the plurality of droplets comprising the cargo is greater than 1 micrometer;
or a combination thereof.

33. (canceled)

34. (canceled)

35. (canceled)

36. The liquid crystal-infused porous surface of claim 21, wherein the anisotropic lubricant further comprises a plurality of droplets comprising a cargo and wherein the release of at least a portion of the plurality droplets comprising the cargo is temperature sensitive as: when the temperature of the thermotropic liquid crystal mesogen is increased from a temperature below the third transition temperature to a temperature above the third transition temperature, then the thermotropic liquid crystal transitions to the isotropic mesophase, such that at least a portion of the plurality of droplets comprising the cargo are automatically released into an aqueous droplet present on the film of anisotropic lubricant.

37. The liquid crystal-infused porous surface of claim 14, wherein:
the anisotropic lubricant further comprises a photoisomerization compound; and
when the photoisomerization compound is a first photoisomer, the third transition temperature has a first value;
when the photoisomerization compound is a second photoisomer, the third transition temperature has a second value;
the first value is greater than the second value;
in the absence of a certain wavelength of light, the photoisomerization compound is the first photoisomer; and
when the photoisomerization compound is exposed to the certain wavelength of light, then the photoisomerization compound isomerizes to the second photoisomer; and
when the anisotropic lubricant is at a temperature above the first value and below the second value, then release of at least a portion of the plurality droplets comprising the cargo is light sensitive as:
in the absence of the certain wavelength of light, the photoisomerization compound is the first photoisomer such that the third transition temperature has the first value, the thermotropic liquid crystal mesogen is in the nematic mesophase, and substantially none of the plurality of droplets comprising the cargo are released from the anisotropic lubricant into an aqueous droplet present on the film of anisotropic lubricant; and
when the photoisomerization compound is exposed to the certain wavelength of light, then the photoisomerization compound isomerizes to the second photoisomer such that the third transition temperature has the second value, the thermotropic liquid crystal mesogen is in the isotropic mesophase, and at least a portion of the plurality of droplets comprising the cargo are released from the anisotropic lubricant into an aqueous droplet present on the film of anisotropic lubricant.

38. (canceled)

39. The liquid crystal-infused porous surface of claim 21, wherein the anisotropic lubricant further comprises a plurality of droplets comprising a cargo and wherein:
when the aqueous droplet has a first zeta potential and the cargo has a second zeta potential, the release of at least a portion of the plurality droplets comprising the cargo when the thermotropic liquid crystal mesogen is in the nematic mesophase is charge sensitive, as:
when the first zeta potential and the second zeta potential have the same sign, then substantially none of the plurality of droplets comprising the cargo are released from the anisotropic lubricant into the aqueous droplet; and
when the first zeta potential and the second zeta potential have opposite signs, then at least a portion of the plurality of droplets comprising the cargo are released from the anisotropic lubricant into the aqueous droplet.

40-66. (canceled)

67. A method of use of the liquid crystal-infused porous surface of claim 1 for drug delivery, organic contaminant removal, heavy metal ion removal, theranostics, biomedical applications, or a combination thereof.

68. A device comprising the liquid crystal-infused porous surface of claim 1, wherein the device is a drug delivery device, a microfluidic device, a smart surface reactor, or a wastewater diagnosis and treatment device.

69-72. (canceled)
